(19) United States
(12) Patent Application Publication
(10) Pub. No.: US 2005/0059846 A1
(43) Pub. Date: Mar. 17, 2005

(54) Process for producing gas clathrate and production apparatus

(76) Inventors: Kazuo Kohda, Kanagawa (JP); Hiroyuki Ida, Kanagawa (JP); Hideyuki Miyamoto, Kanagawa (JP)

Correspondence Address: FRISHAUF, HOLTZ, GOODMAN & CHICK, PC, 767 THIRD AVENUE, 25TH FLOOR, NEW YORK, NY, 10017-2023, US

(21) Appl. No.: 10/501,299

(22) PCT Filed: Nov. 29, 2002
(86) PCT No.: PCT/JP02/12496

(30) Foreign Application Priority Data
Sep. 11, 2002 (JP) 2002-265079
Sep. 11, 2002 (JP) 2002-265080
Sep. 13, 2002 (JP) 2002-267526
Sep. 18, 2002 (JP) 2002-271225
Sep. 19, 2002 (JP) 2002-272901

(51) Int. Cl. C07C 9/00; G05D 7/00; G05D 16/00

(52) U.S. Cl. 585/15; 422/224; 422/198; 422/110; 422/112

(57) ABSTRACT

A method for producing gas clathrate provides a mixing/dissolving step, where raw material liquid and raw material gas are mixed on the way of the line and where the raw material gas is dissolved into the raw material liquid. The method, furthermore, provides a forming step, where the gas clathrate is formed by cooling the raw material liquid, into which the raw material gas has been dissolved, while forcing the raw material liquid to flow through a reaction conduit. An apparatus for producing gas clathrate provides a line mixer, where the raw material liquid and the raw material gas are mixed on the way of the line, and where the raw material gas is dissolved into the raw material liquid. And, the apparatus provides a reaction conduit for forming the gas clathrate by cooling, simultaneously with forcing the raw material liquid to flow through the reaction conduit.

### FIELD OF THE INVENTION

The present invention relates to a method for producing gas clathrate through the reaction between liquid and raw material gas such as natural gas and to an apparatus therefor. In case that a host substance is liquid, the substance is defined as gas clathrate. In case that a host substance is water, the substance is defined as gas hydrate.

### BACKGROUND OF THE INVENTION

Gas hydrate is an ice-like material having a cage structure formed by water molecules, whose cage keeps high concentration of gas molecules such as those of natural gas and carbon dioxide therein. Gas hydrate can keep a large amount of the gas per unit volume, and can store and transport the gas at comparatively high temperature under the atmospheric pressure, compared with liquefied natural gas. Therefore, it has been taken a notice to apply the natural gas hydrate and the like to the transportation and to the storage.

Conventionally, concerning naturally existing gas hydrate (so-called methane hydrate), the studies have been focused on the utilization. However, in these recent years, the concerned studies are making attempt to generate the gas hydrate in the industrial world, paying attention to such a characteristic of the gas hydrate.

The conventional process for producing the gas hydrate has been done as follows, as the outline. Water and raw material gas such as natural gas get in contact with each other to dissolve the raw material gas into the water. In this case, the temperature of the gas and the water and the pressure of these ones are kept within a range of forming the gas hydrate. This range is led by an equilibrium curve of the gas hydrate. The formed gas hydrate, which is so-called in a sherbet state, is separated and de-watered from unreacted gas and form raw material water. Furthermore, the gas hydrate is subjected to various treatments such as freezing and forming. And then, the formed gas hydrate is stored in a storing facility. Afterwards, the gas hydrate is taken out from the storing facility to be transported to, in accordance with the necessary demand.

In the process of producing the gas hydrate, the most important factors to control the generation rate of the gas hydrate are as follows. These are the diffusion rate and the dissolving rate of the gas into the water, and the efficiency of depriving the reaction heat that generates by the reaction between the gas and the water.

There is one technological example that produces the gas hydrate efficiently, which is obtained by increasing the dissolving rate of the gas into the water and by increasing the heat deriving efficiency while forming the gas hydrate. JP-A-2001-10985, (the term “JP-A” referred herein signifies the “unexamined Japanese patent publication”), shown in FIG. 6, discloses an apparatus and a method for producing natural gas hydrate.

According to the disclosure, several gas hydrate producing units A, B, D are connected with mutually in series, whose number of the units is determined by the gas composition of the applied natural gas, respectively. The respective gas hydrate producing units have a pressure vessel 51, which contains a porous plate 55 that divides the-pressure vessel 51 into a gas space 56 and into a gas/liquid contact space 52. The respective gas hydrate producing units, also, have a coil evaporator 53, which is located in two or more stages in the gas/liquid contact space 52. Furthermore, the respective gas hydrate producing units have the following devices. These are to say, a refrigerator 58 that supplies refrigerant to the coil evaporators 53, a gas hydrate storage tank 62 that is connected with the exit of the gas/liquid contact space 52 via a buffer tank 59, a raw material water feed pipe 61 that feeds the raw material water at bottom of the storage tank 62 to the bottom part of the gas/liquid contact space 52. Furthermore, the respective gas hydrate producing units have a raw material gas feed pipe 57 that feeds the natural gas to the gas space 56. Every storage tank 62 has a gas discharge pipe 70 at the upper space thereof, whose gas discharge pipe 70 is connected with a recovered gas mixer 66 in the downstream side of the storage tank 62.

The above-described conventional technology has, however, the following various problems.

As described above, the factors to control the forming-rate are the diffusion-rate of the gas into the water are the dissolving-rate of the gas into the water, and the efficiency of deriving the reaction heat generated by the reaction between the gas and the water.

From this viewpoint, in order to enhance the gas to diffuse and dissolve into the water, the above-described conventional technology adopts the porous plate 55, which generates fine gas bubbles to increase the mutual contact area between the water and the gas.

However, the method for introducing bubbles by using the porous plate 55 makes it impossible to create the size of the fine bubbles sufficiently. So, the effective result is unexpected, such as enhancing the gas dissolution by widening the mutual contacted area between the gas and the liquid.

Furthermore, there is required to prepare a space for allocating the porous plate 55 having the equal or more than the determined area. And there is required to secure a mutual contact-space between the gas and the liquid 52, whose space is the equal or more than the determined area, which has a role for the gas to get contact with the liquid in the pressure vessel 51. Consequently, there occurs a problem that the volume of the pressure vessel 51 should be much bigger, resulting in inviting a facility problem.

In addition, there becomes a possibility for the hydrate to adhere to the porous plate 55 and to grow on the porous plate. Such a possibility ends up in clogging the pores on the plate, if the worst case happens.

It is also one of the most important factors to derive the generated reaction heat, accompanied with forming the gas hydrate. From the standing point of view, a higher capacity of the pressure vessel 51 as the reactor has a disadvantage, which is, it is not enough to cool merely the wall surface of the pressure vessel.

In order to take a countermeasure against the problem, the above-mentioned example of the conventional technology adopts a means for providing refrigerant circulation coils 53 inside the pressure vessel 51. But, such means invite a problem to increase in the size and the complexity of the facility.

There is another problem in the conventional technology. That is, when the gas hydrate is formed in a pressure vessel, the formed gas hydrate floats on the water in the pressure vessel. Accordingly, there is required to prepare a mean for taking out the floating gas hydrate from the pressure vessel. (For example, a discharge opening for the mixture of the gas hydrate and the water, and a unit to control the water level). This problem also makes the facility complex.

As mentioned above, the conventional technology has invited such a problem that there has been required for the complex facility and for the expanded facility, up to now.

### SUMMARY OF THE INVENTION

It is an object of the present invention to provide a method for producing gas clathrate and an apparatus therefor, whose method and apparatus efficiently enables the gas to dissolve into liquid and enables the reaction heat to be derived. And, the method and apparatus makes it possible to prepare a simple and compact apparatus.

In order to attain the object, the present invention provides a method for producing gas clathrate, comprising mixing and dissolving step of mixing a raw material liquid with a raw material gas on the way of the line for producing the gas clathrate and dissolving the raw material gas into into the raw material liquid, and forming step of cooling the gas-dissolved raw material liquid while the gas-dissolved raw material liquid flows through a reaction conduit to form the gas clathrate.

The mixing and dissolving step preferably comprises continuously dissolving the raw material gas in the form of fine bubbles into the raw material liquid.

It is preferable that the mixing and dissolving step comprises, without using a reaction vessel, mixing a raw material liquid with a raw material gas in a production line of gas clathrate and dissolving the raw material gas into the raw material liquid without using a reaction vessel and the forming step comprises, without using a reaction vessel, cooling the gas-dissolved raw material liquid while the gas-dissolved raw material liquid flows through the reaction conduit to form the gas clathrate.

It is preferable that the mixing of the raw material liquid and the raw material gas is continuously performed by using a line mixer.

It is preferable that the mixing and dissolving step comprises mixing a raw material liquid with a raw material gas by using a line mixer and dissolving the raw material gas into the raw material liquid, and the forming step comprises forcing the gas-dissolve raw material liquid to flow through a pipe-shaped reaction conduit and cooling the surface of the reaction conduit to form the gas clathrate.

When the line is used, a pressure regulating means is preferably located between the line mixer and the reaction conduit. It is preferable that the method of the present invention further comprises the step of adjusting a fluid pressure such that the pressure of line mixer side becomes higher than the pressure of the reaction conduit side. Also, the method of the present invention further comprises the step of adjusting a flow speed of fluid to reduce the flow speed flowing through the line by a flow speed regulating means located at the downstream side of the line mixer.

The method for producing gas clathrate according to the present invention may comprise further mixing and dissolving step of dissolving a raw material gas into the raw material liquid after the mixing and dissolving step and before the step of forming the gas clathrate, or on the way of the forming step.

In the method for producing the gas clathrate according to the present invention, the mixing and dissolving step and the forming step may be separately performed. In such a case, the mixing and dissolving step preferably comprises mixing a raw material liquid with a raw material gas by using a line mixer in the production line of the gas clathrate and dissolving the raw material gas into the raw material liquid. And the forming step preferably comprises forcing the gas-dissolved raw material liquid to flow through a pipe-shaped reaction conduit in order to form the gas clathrate.

In the method for producing the gas clathrate according to the present invention, it is preferable that the forming step comprises converting all of the raw material gas dissolved in the mixing and dissolving step to the gas clathrate.

It is preferable that the forming step is performed on the following condition:
(a) The pressure P at exit of the reaction conduit becomes higher than the minimum pressure P₀ for forming the gas clathrate.
(b) The temperature T in the reaction conduit becomes lower than the maximum temperature T₀ for forming the gas clatharte, and,
(c) The flow rate of the raw material liquid, the pressure of the raw material liquid, the flow rate of the raw material gas, the pressure of the raw material gas, the cooling capacity, the reaction conduit length and the reaction conduit diameter are determined to be in the forming step so that the raw material gas dissolved in the mixing and dissolving step absorb all of the formed heat, where all of the dissolved raw material gas is conveted to the gas clathrate.

It is preferable that the method for producing gas clatharte further comprises changing a particle size of the gas clathrate by changing at least one of the flow speed of the raw material liquid flowing through the reaction conduit and the flow rate of the raw material gas. In case of a plurality of reaction conduits, the producing step further comprises changing sizes of the gas clathrate formed in the respective reaction conduits by changing at least one of the flow speeds of the raw material liquid flowing through each of a plurality of the reaction conduits and the flow rates of the raw material gas being fed to the respective reaction conduits.

It is desirable that the method for producing the gas clathrate further comprises the step of: sending the formed gas clathrate together with the unreacted raw material gas and the raw material liquid through the reaction conduit to a separator; and separating the mixture into the gas clathrate, the unreacted raw material gas and the raw material liquid. Moreover, the producing method further comprises the step of recycling the unreacted raw material gas and the raw material liquid, which were separated in a separating step, led to the line mixer. In the separator, the liquid level is controlled to have the seal effect of the raw material liquid so as the gas not to enter the raw material liquid not to enter the raw material liquid recycling line.

It is desirable that the method for producing the gas clathrate further comprises the steps of: sending the formed gas clathrate together with the unreacted raw material gas and the raw material liquid through the reaction conduit to a separator; subjecting a slurry including the gas clathrate, the unreacted raw material gas and the raw material liquid to a separation and de-watering treatment by the separator to form a high concentration slurry or a solid clathrate.

In the method having the steps of separating the formed gas clathrate in a separator connected with the reaction conduit, the method further comprises the steps of: detecting pressure in the separator; and regulating the pressure in the separator by adjusting at least one of the flow rate of the raw material gas being fed into the mixing and dissolving step and the flow speed of raw material liquid in the forming step, based on the pressure detected in the pressure detecting step.

Further, the present invention provides an apparatus for producing gas clathrate, comprising: a line mixer for mixing a raw material liquid with a raw material gas and dissolving the raw material gas into the raw material liquid in the production line of the gas clathrate; and a reaction conduit, through which the gas-dissolved raw material liquid to form the gas clathrate.

In the apparatus for producing the gas clathrate, a plurality of reaction conduits are usable.

It is preferable that the line mixer is one for generating fine bubbles of the raw material gas.

It is preferable that the apparatus for producing the gas clathrate further comprises a pressure regulating means at the downstream side of the line mixer to adjust the line pressure.

It is also preferable that the apparatus further comprises flow speed regulating means to adjust flow speed of the fluid flowing through the line.

The apparatus for producing the gas clathrate has no tank-shape pressure vessel for mixing the raw material liquid with the raw material gas and dissolving the raw material gas in the raw material liquid and for cooling the gas-dissolved raw material liquid.

It is desirable that the apparatus for producing the gas clathrate further comprises as follows:
(a) a gas flow rate regulating means for adjusting the flow rate of the raw material gas to be fed;
(b) gas flow pressure regulating means for adjusting the raw material gas pressure;
(c) raw material liquid flow rate regulating means for adjusting the pressure of the raw material liquid;
(d) raw material liquid pressure regulating means for adjusting the pressure of the raw material liquid;
(e) a cooling unit which cools the reaction conduit; and
(f) pressure regulating means for adjusting the pressure in the reaction conduit.

In the apparatus having the above-mentioned (a)-(f), the gas flow rate regulating means, the gas pressure regulating means, the raw material liquid flow rate regulating means, the raw material pressure regulating means, the cooling capacity of the cooling unit, the length of the reaction conduit, and the diameter of the reaction conduit are determined to be under the condition that all of the raw material gas supplied into the line mixer are converted to the gas clathrate.

Also, in the apparatus having the above (a)-(f), the gas flow rate regulating means, the gas pressure regulating means, the raw material liquid flow rate regulating means, the raw material liquid pressure regulating means, the cooling capacity of the cooling unit, the length of the reaction conduit, and the diameter of the reaction conduit are determined to be under the conditions that the pressure P at the exit of the reaction conduit becomes higher than the minimum pressure P₀ for forming the gas clathrate. And, the temperature T in the reaction conduit becomes lower than the maximum temperature T₀ for forming the gas hydrate so that the raw material gas supplied into the line mixer absorbs the formation heat, where all of the dissolved raw material gas are converted to the gas clathrate.

It is preferable that the apparatus for producing the gas clathrate further comprises a flow speed controlling means for changing the flow speed of the raw material liquid flowing through the reaction conduit. In case of a plurality of reaction conduits, the apparatus further comprises flow speed controlling means for changing the flow speed of the raw material water flowing through the plurality of reaction conduits, wherein the flow speed controlling means controls so as the flow speed of the raw material liquid flowing through the individual reaction conduits to be different from each other.

It is preferable that the apparatus for producing gas clathrate further comprises a gas flow rate regulating means for adjusting flow rate of the raw material gas to be fed into the line mixer. In the apparatus having the plurality of the line mixers and the plurality of the reaction conduits, the plurality of the line mixers has gas flow rate-regulating means respectively. The gas flow rate regulating means controls that the flow rate of the raw material gas flowing through the individual reaction conduits differ from each other.

It is preferable that the apparatus for producing gas clathrate further comprises a separator for separating the gas clathrate, the unreacted raw material gas and the raw material liquid, which are formed in the reaction conduit. The separator is one selected from the group consisting of decanter, cyclone, centrifugal, separator, belt press, screw concentrator dehydrator, and rotary drier.

The apparatus having the separator further comprises the following contents:
(a) a gas flow rate regulating means for adjusting a material gas flow rate to be fed into:
(b) a pressure detecting means for detecting a pressure in the separator; and
(c) a control means for adjusting at least one of the gas flow rate of the gas flow regulating means and the flow speed of the raw material liquid flow speed regulating means based on the pressure detected by the pressure detecting means.

In the apparatus for producing gas clathrate, the line mixer is at least one of line mixer arranged upstream side of the reaction conduit and at least one line mixer is further arrange on the way of the reaction conduit.

### BRIEF DESCRIPTION OF THE DRAWING

FIG. 1 is a schematic drawing of an apparatus for producing gas hydrate according to Embodiment 1.

FIG. 2 is an explanation view of a line mixer.

FIG. 3 is a schematic drawing of another apparatus for producing gas hydrate according to Embodiment 1.

FIG. 4 is a schematic drawing of another apparatus for producing gas hydrate according to Embodiment 1.

FIG. 5 is an explanation view of a method for producing gas hydrate according to Embodiment 1.

FIG. 6 is a schematic drawing of an apparatus for producing natural gas hydrate in the prior art.

FIG. 7 is a schematic drawing of an apparatus for producing gas clathrate according to Embodiment 2.

FIG. 8 is an explanation view to illustrate the mechanism of total conversion to hydrate in the reaction conduit according to Embodiment 2.

FIG. 9 is a schematic drawing of another apparatus for producing gas clathrate according to Embodiment 2.

FIG. 10 is a schematic drawing of another apparatus for producing gas clathrate according to Embodiment 2.

FIG. 11 is a schematic drawing of an apparatus for producing gas clathrate according to Embodiment 2.

FIG. 12 is a schematic drawing of an apparatus for producing gas hydrate according to Embodiment 3.

FIG. 13 is a schematic drawing of another apparatus for producing gas hydrate according to Embodiment 3.

FIG. 4 is a schematic drawing of another apparatus for producing gas hydrate according to Embodiment 3.

FIG. 15 is a schematic drawing of an apparatus for producing gas hydrate according to Embodiment 3.

FIG. 16 is a schematic drawing of an apparatus for producing gas clathrate according to Embodiment 4.

FIG. 17 is a schematic drawing of an apparatus for producing gas clathrate according to Embodiment 4.

FIG. 18 is a schematic drawing of an apparatus for producing gas hydrate according to Embodiment 5.

FIG. 19 is a schematic drawing of another apparatus for producing gas hydrate according to Embodiment 5.

FIG. 20 is a schematic drawing of an apparatus for producing gas hydrate according to Embodiment 5.

FIG. 21 is a schematic drawing of an apparatus for producing gas hydrate according to Embodiment 5.

FIG. 22 is a schematic drawing of an apparatus for producing gas hydrate according to Embodiment 6.

### EMBODIMENT FOR CARRYING OUT THE INVENTION

### Embodiment 1

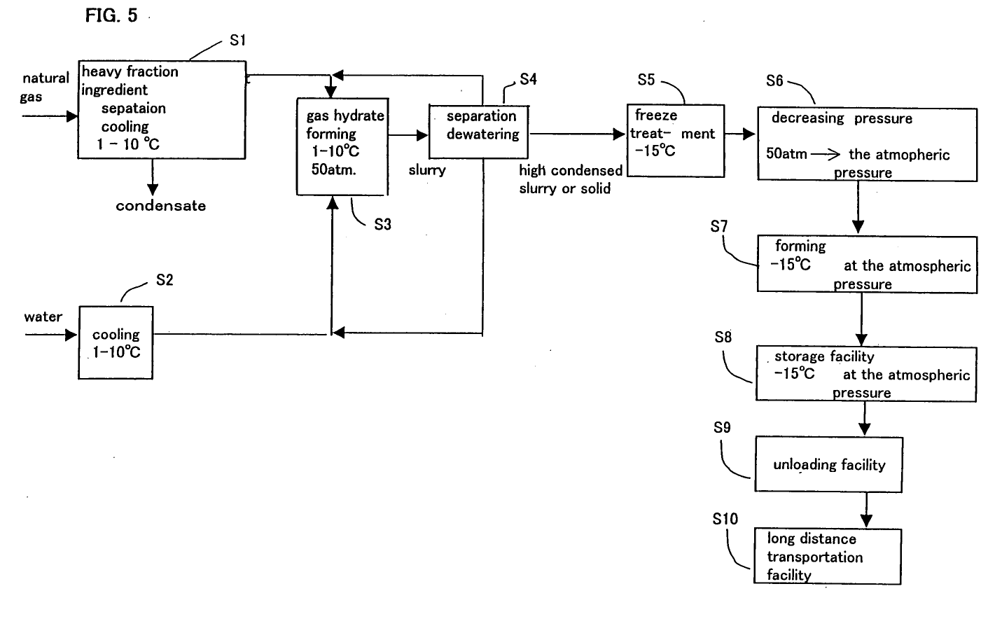

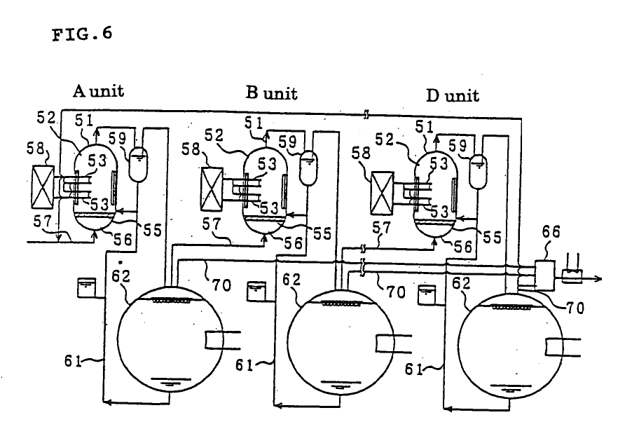

FIG. 5 shows a schematic view for explaining a process for producing gas hydrate according to Embodiment 1 of the present invention. The process uses natural gas as the raw material gas. The process for producing gas hydrate is outlined referring to FIG. 5.

The natural gas is cooled to temperatures of 1° C. to 10° C. to separate heavy fraction thereof as a condensate, (S1). On the other hand, water is also cooled to temperatures of 1° C. to 10° C., (S2). The cooled water and the cooled natural gas react with each other under the condition of 1° C. to 10° C. and 50 atm to form the gas hydrate, (S3). The formed gas hydrate in slurry state is treated by separation and by de-watering to form heavy slurry or solid, (S4). The separated water and the unreacted gas are recycled to the reaction step (S3).

After separated and dewatered, the gas hydrate is subjected to freeze treatment at temperatures around −15° C., (S5). The freeze treatment is given to stabilize the gas hydrate by freezing the water attached to the surface of gas hydrate after separated and dewatered, thus forming an ice shell on the surface thereof.

After the freeze treatment, the gas hydrate is subjected to depressurizing treatment, or decreasing the pressure from 50 atm to atmospheric pressure, (S6). Afterwards, the freeze-treated gas hydrate is formed to pellets, (S7), and is stored at a storage facility such as silo, (S8), and then, is transferred, in accordance with the requirement, by an unloading facility such as belt conveyers, (S9). It may be a case, for a long distance transportation, a transportation facility such as tanker (S10) is used.

That is the outline of the gas hydrate producing process. The preferred embodiment according to the present invention gives an improvement in the step (S3) that forms slurry gas hydrate from water and natural gas. The detail of the improvement is described as follows.

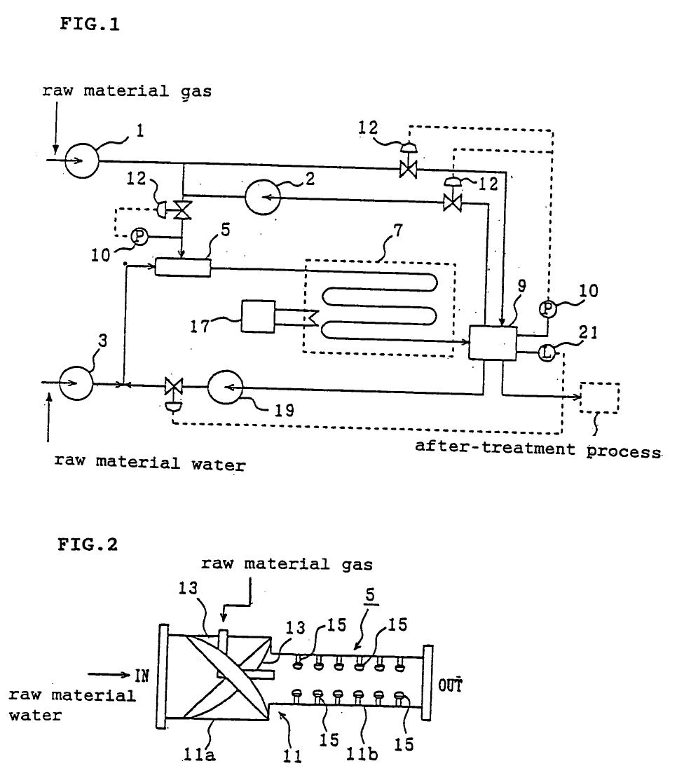

FIG. 1 shows a process diagram of the Embodiment according to the present invention, giving main units. The units of the Embodiment according to the present invention are described referring to FIG. 1.

The gas hydrate producing apparatus of Embodiment 1 has gas boosters 1, 2 that increase the pressure of raw material gas such as natural gas, raw material water pumps 3, 19 that feed the raw material water. And, the apparatus has a line mixer 5 that mixes the raw material water with the raw material gas to dissolve the raw material gas in the raw material water. And, the apparatus has a reaction conduit 7 that cools the mixture formed by the line mixer 5 to form the gas hydrate, and a separator 9 that separates the gas hydrate formed in the reaction conduit 7, unreacted gas, and the raw material water from each other.

These units are connected to each other by pipes given in the drawing with solid lines with arrow marks. Pressure detectors 10 are located at key points in the pipeline. Each of the valves 12 positioned at key points is controlled by the signals transmitted from the respective pressure detectors 10, thus adjusting the pressure and the flow rate of the respective pipes.

Further detailed description is given below for critical units in the line.

The line mixer 5 in Embodiment 1 has a cylinder 11 of two-stage structure. Here, the inlet has a large diameter, and the outlet has a small diameter, as illustrated in FIG. 2, (cited from page 7 of catalogue of “OHR line mixer” of Seika Corp.) The cylinder 11 has blades 13 called the guide vanes in the large diameter section 11a. The cylinder has a plurality of impingers 15 in mushroom shape extending from the inner periphery of the cylinder toward the center thereof in the small diameter section 11b at downstream side of the large diameter section 11a.

In such a type of line mixer 5, the raw material water that is fed by the raw material water pump 3 there into, creates a swirling flow caused by the blades 13. The resulted strong centrifugal force pushes the raw material water outward, whose raw material water then collides against the mushroom impingers 15 to receive stronger agitation. The raw material gas is caught by the vigorous agitation of the raw material water to form very fine bubbles, thus the raw material water and the raw material gas are mixed together. As a result, the contact area between the raw material gas and the raw material water increases to assure efficient dissolution of the raw material gas in the raw material water.

A single or plurality of the curved pipes structures the reaction conduit 7. The chiller 17 cools the surface of the reaction conduit 7. Such a reaction conduit 7 makes it possible to cool from periphery efficiently. As a result, there becomes no need to cool directly the gas and the raw material water by cooling the coil or other means. The direct cooling is applied to the conventional method, though. Consequently, this embodiment enables the conformation of the facility to become simple and compact.

In advance, the line mixer 5 mixes and dissolves the raw material gas and the raw material water. Then, the process allows the reaction conduit 7 to exclusively concentrate on cooling. Such a process-concept makes it possible to use this type of reaction conduit 7. That is to say, mixing and dissolving the raw material gas and the raw material water, and cooling the reaction are conducted in a tank-shape pressure vessel, according to the conventional method. Here, the mixing and dissolving action require a high volume of a space. Such a high volume of the space unables the process to cool solely from periphery of the reaction tank. On the contrary, according to Embodiment 1 of the present invention, mixing and dissolving the raw material gas and the raw material water are done, separated from cooling the reaction. So, in the reaction step, cooling is exclusively concentrated. Such a process makes it possible to cool by a simple structure, as described above.

The separator 9 separates gas hydrate, unreacted gas, and raw material water from each other. Applicable separator 9 includes decanter, cyclone, centrifugal separator, belt press, screw concentrator dehydrator, and rotary drier.

The process for producing gas hydrate by using a facility of Embodiment 1, which has a structure mentioned above, is described in detail, as follows.

The pressure of raw material gas increases to a determined level by the gas booster 1. The pressure of the raw material water, also, increases to a determined level by the raw material water pump 3. Both the pressurized raw material gas and the pressurized raw material water are fed into the line mixer 5. The pressurized raw material gas and the pressurized raw material water, which are fed into the line mixer 5, are vigorously mixed together under the mechanism described before. At that time, the raw material gas becomes fine bubbles to enter the raw material water and to mix with the raw material water, thus enhancing the raw material gas to dissolve into the raw material water.

The raw material water with the dissolved raw material gas, (which also contains the undissolved fine bubbles), is fed into the reaction conduit 7, where the mixture is cooled by the chiller 17 to form the gas hydrate. The formed gas hydrate, along with the unreacted gas and the raw material water, flows through the conduit to enter the separator 9.

According to Embodiment 1, the reaction between the raw material gas and the raw material water is carried out while letting them move through the conduit, so the gas hydrate forming step sends all the materials (formed gas hydrate, unreacted gas, and raw material water) to the separator 9. That is, there is no need to take out only the formed gas hydrate, which the conventional method needed for. And the constitution of the facility becomes simple.

The mixture of the gas hydrate, the unreacted gas, and the raw material water, which is fed into the separator 9, is separated into the gas hydrate, the unreacted gas, and the raw material water, respectively. The separated raw material water is recycled to the line mixer 5 by the pump, 19. The pressure of the unreacted raw material gas increases to a determined level by the gas booster 2, in order to feed into the line mixer 5.

The formed gas hydrate is taken out from the separator 9, and is fed into the succeeding process (S5 and following processes in FIG. 5).

In the separator 9, a level gauge 21 detects the water level, and the water level in the separator 9 is controlled to be at the equal or higher determined level. The reason why is that the raw material water has the same effect as a seal-water, so as the gas not to enter recycle line of the raw material water. The pressure of the raw material water unnecessary for the water seal increases to a determined level by the raw material water pump 19, and is fed into the line mixer 5.

The gas booster 1 feeds the pressurized raw material gas directly to the separator 9, in order to keep the internal pressure of the separator 9 to a predetermined level or higher.

As described above, according to Embodiment of the present invention, the line mixer 5 in cylindrical shape continuously does dissolving the raw material gas in raw material water, thus the dissolution is conducted in a space-saving and efficient manner.

Furthermore, dissolving the raw material gas in the raw material water by the line mixer 5 that is separated from the reactor allows the pipe-shape reaction conduit 7 to be usable, instead of the conventional reactor. This makes it possible to apply a simple and a compact cooling means that cools the peripheral surface of the conduit.

In addition, both of them, which mean, dissolving the raw material gas in the line mixer 5 and forming the gas hydrate in the reaction conduit 7, are done continuously, so that the efficiency for producing the gas hydrate increases drastically.

According to the above Embodiment of the present invention, there has been described no means for adjusting the pressure between the line mixer 5 and the reaction conduit 7.

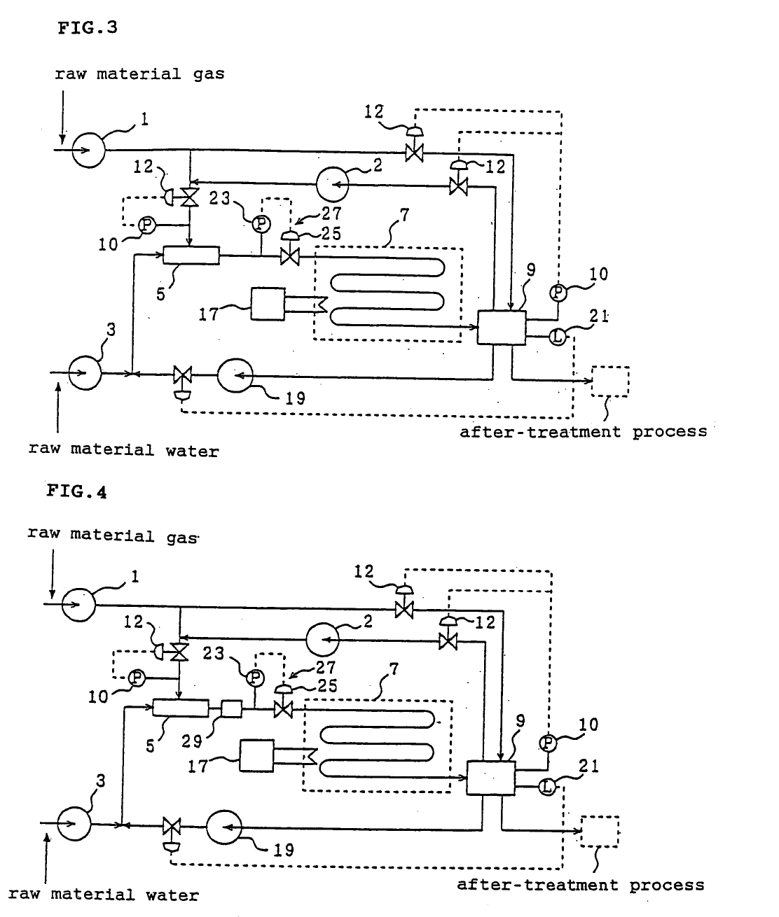

As shown in FIG. 3, however, a pressure regulating means 27 having a pressure detector and a pressure regulating valve 25 may be located between the line mixer 5 and the reaction conduit 7.

The pressure regulating means 27 makes it possible to increase the pressure on the side of the line mixer 5, resulting in enhancing the raw material gas to dissolve into the raw material water in the line mixer 5.

In order to enhance further more the raw material gas to dissolve into the raw material water, there may be a case, shown in FIG. 4. That is, a holding section 29 as the flow speed regulating means for decreasing the flow speed of the fluid flowing through the line may be located at downstream side of the line mixer 5. Applying the holding section 29 enables the raw material gas, which became fine bubbles in the line mixer 5, to save a time for dissolving in the raw material water. This invites the embodiment to enhance the raw material gas to dissolve into the water.

As one of the example of the holding section 29,there may be a tank having a determined volume.

The above-given description does not indicate the details of temperature and pressure in the individual steps. As one example, there may be the one given in FIG. 5. The temperature and the pressure at each step are selected to the respective optimum values.

The above-given Embodiment according to the present invention deals with natural gas consisting mainly of methane gas as the raw material gas. Other applicable examples of the raw material gas include ethane, propane, butane, krypton, xenon, and carbon dioxide.

AS further examples of the line mixer, there may be a case, another type of the line mixer such as Venturi type, which has a thinning portion in the middle of the cylinder to generate negative pressure for sucking and mixing the raw material gas. Or there may be a case, another type such as in cone or truncated cone shape to create swirling flow therein to conduct mixing of gas and liquid. One example of the latter type mixer is a swirling type fine bubbles generator disclosed in JP-A-2000-447. In short, the line mixer according to Embodiment of the present invention broadly includes the unit which is located in the line and which is continuously able to mix the gas with liquid.

According to the above-given Embodiment for carrying out the present invention, the reaction conduit 7 may be single or a plurality of curbed pipes, as an example. The reaction conduit 7 may be formed by a plurality of straight pipes.

### Embodiment 2

The method for producing gas hydrate according to Embodiment 2 has the steps of: mixing the raw material water with the raw material gas and dissolving the raw material gas into the raw material water in the route for producing the gas hydrate; and cooling thus mixed and dissolved mixture while the mixture flows through a reaction conduit to form the gas hydrate. The gas hydrate-forming step is determined to be under the conditions of a flow rate of the raw-material water, a pressure of the raw material water, a flow rate of the raw material gas, a pressure of the raw material gas, a cooling capacity, a reaction conduit length, and a reaction conduit diameter so as the pressure P at exit of the reaction conduit to become higher than the minimum pressure P₀ for forming the gas hydrate and so as the temperatures T in the reaction conduit to become lower than the maximum temperature T₀ for forming the gas hydrate, and so as the raw material gas, which has been mixed and dissolved in the above-mentioned mixing/dissolving process to deprive all of the heat generated by all amount of the clathrate.

The apparatus for producing gas according to Embodiment 2 has: a gas flow rate regulating means for adjusting the flow rate of the feeding raw material gas; a gas pressure regulating means for adjusting the raw material gas pressure; a raw material water flow rate regulating means for adjusting the flow rate of the feeding raw material water; a raw material water pressure regulating means for adjusting the pressure of the raw material water; a line mixer which mixes the raw material water with the raw material gas and which dissolves the raw material gas into the raw material water in the route for producing the gas hydrate; a reaction conduit which cools the raw material water mixed with the raw material gas and containing the dissolved raw material gas while the mixture flows therethrough; a cooling unit which cools the reaction conduit; and pressure regulating means for adjusting the pressure in the reaction conduit. The gas flow rate regulating means, the gas pressure regulating means, the raw material water flow rate regulating means, the raw material pressure regulating means, cooling capacity of the cooling unit, length of the reaction conduit, and diameter of the reaction conduit are determined to be under the conditions so as the pressure P at exit of the reaction conduit to become higher than the minimum pressure P₀ for forming the gas hydrate and so as the temperatures T in the reaction conduit to become lower than the maximum temperature T₀ for forming the gas hydrate, and so as the raw material gas supplied to the line mixer to deprive the heat generated by all amount of the clathrate.

The apparatus for producing gas hydrate according to Embodiment 2 further has a pressure detector for detecting the pressure at exit of the reaction conduit in order to adjust one or both of the gas flow rate regulating means and the raw material water flow rate regulating means when the pressure detected by the pressure detector exceeds a predetermined level.

The line mixer is a device for generating fine bubbles of the raw material gas.

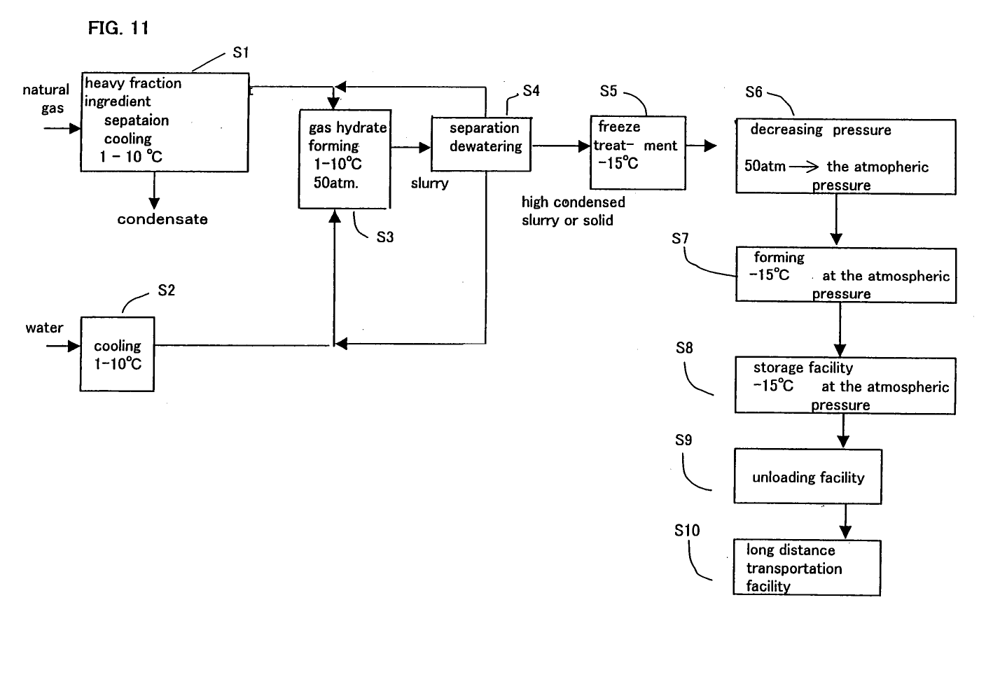

Gas hydrate, which is one Embodiment of gas clathrate, are described, as follows. * FIG. 11 shows an outline of the gas hydrate producing process according to Embodiment 2. The process uses natural gas as the raw material gas. The process for producing gas hydrate is outlined referring to FIG. 11.

Embodiment 2 shows an improvement in the step (S3) that forms slurry gas hydrate from water and natural gas, where the total amount of the fed raw material gas is converted to hydrate, thus attaining the same composition between the raw material gas consisting of a plurality of gases and the formed hydrate. The improvement is described in details, as follows.

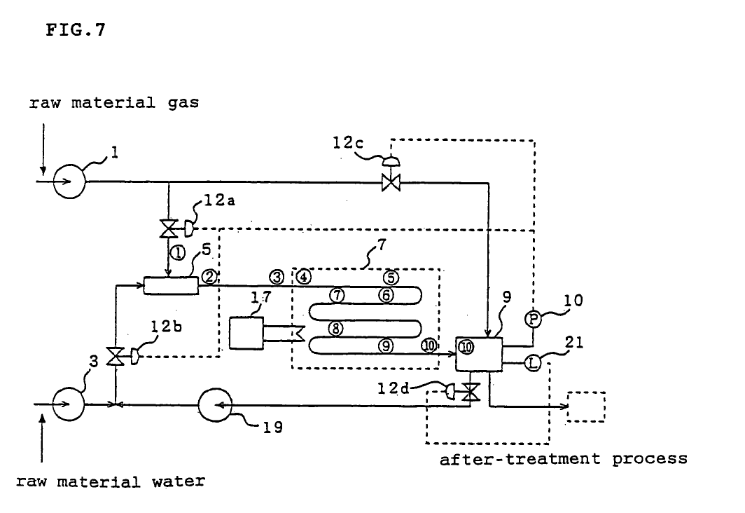

FIG. 7 shows a process diagram of Embodiment 2 for carrying out the invention, giving main units. The units of the Embodiment 2 are described referring to FIG. 7.

The gas hydrate producing apparatus of the Embodiment 2 has a gas booster 1 (corresponding to the gas pressure regulating means of the Embodiment 2) which increases the pressure of raw material gas such as natural gas, raw material water pumps 3, 19 (corresponding to the raw material water pressure regulating means of the Embodiment 2) which pressurize and feed the raw material water, a line mixer 5 which mixes the raw material water with the raw material gas to dissolve the raw material gas in the raw material water, a reaction conduit 7 which cools the mixture formed by the line mixer 5 to form the gas hydrate, a chiller 17 which cools the reaction conduit 7 as the cooling unit, and a separator 9 which separates the gas hydrate formed in the reaction conduit 7 and the raw material water from each other.

These units are connected to each other by pipes given in the drawing with solid lines with arrow marks. A pressure detector 10 is mounted to the separator 9. Based on the signals transmitted from the pressure detector 10, a valve 12a (corresponding to the gas flow rate regulating means of the Embodiment 2), a valve 12b (corresponding to the raw material water flow rate regulating means of the Embodiment 2), and a valve 12c (corresponding to the gas pressure regulating means of the Embodiment 2) on the respective pipelines are controlled, thus adjusting the pressure and the flow rate of on the respective pipelines.

With the constitution described above, the valves 12a, 12b, 12c, the gas booster 1, the raw material water pumps 3, 19, the cooling capacity of the chiller 17, the length of the reaction conduit 7, and the diameter of the reaction conduit 7 are determined to be so as the pressure P in the separator 9, (corresponding to the pressure at exit of the reaction conduit 7) to become higher than the minimum pressure P₀ for forming hydrate, and so as the temperatures T in the reaction conduit 7, (temperature at every point in the reaction conduit 7), to become lower than the maximum temperature T₀ for forming hydrate.

Further detailed description is given below for critical units in the line.

The line mixer 5 in the Embodiment 2 has a cylinder 11 of two stage structure giving a large diameter at inlet and a small diameter at exit, which is illustrated in FIG. 2, (cited from page 7 of catalogue of “OHR line mixer” of Seika Corp.) The cylinder 11 has blades 13 called the guide vanes in the large diameter section 11a, and has a plurality of impingers 15 in mushroom shape extending from the inner periphery of the cylinder toward the center thereof in the small diameter section 11b at downstream side of the large diameter section 11a.

In such a type of line mixer 5, the raw material water which is fed by the raw material water pump 3 thereinto creates a swirling flow caused by the blades 13. The resulted strong centrifugal force pushes the raw material water outward, which raw material water then collides against the mushroom impingers 15 to receive stronger agitation. The raw material gas is caught by the vigorous agitation of the raw material water to form very fine bubbles, thus the raw material water and the raw material gas are mixed together. As a result, the contact area between the raw material gas and the raw material water increases to assure efficient dissolution of the raw material gas in the raw material water.

The reaction conduit 7 is structured by a single or plurality of curved pipes. The chiller 17 cools the surface of the reaction conduit 7. The reaction conduit 7 makes it possible to cool the process from periphery, efficiently. As a result, there is no need in cooling directly the gas and raw material water by cooling coil or other means, whose direct cooling is applied to the conventional method disclosed in the Patent document 1 and the Patent document 2, thus the constitution of the facility becomes simple and compact.

In advance, the type of reaction conduit 7 is applied to realize that raw material gas and the raw material water in the line mixer 5 are mixed and dissolved. And such an application allows the reaction conduit 7 to exclusively concentrate on the cooling. That is, according to the conventional method described in the Patent document 1 and the Patent document 2, the mixing, dissolving, and cooling the reaction of the raw material gas and the raw material water are conducted in a tank-shape pressure vessel. So the mixing and dissolving action requires a determined volume of a space, which makes it impossible to cool solely from periphery of the reaction tank. On the contrary, according to Embodiment 2, mixing and dissolving the raw material gas and the raw material water are separated from cooling the reaction, so the reaction step enables the process to concentrate on the cooling. As a result, cooling such as simple constitution described above is applied to the embodiment.

The separator 9 separates mainly the gas hydrate from the raw material water. The Applicable separator 9 includes decanter, cyclone, centrifugal separator, belt press, screw concentrator dehydrator, and rotary drier.

The pressurized raw material gas is fed into the separator 9. Owing to the pressure of the raw material gas, the internal pressure of the separator 9 is kept above the minimum pressure P₀ for forming hydrate. Adjusting the internal pressure of the separator 9 to above P₀, enables the internal pressure of the reaction conduit 7, which is positioned at upstream-side of the separator 9, to become higher than P₀.

The process for producing gas hydrate using a facility of the Embodiment 2 having the constitution given above is described as follows.

The pressure of the raw material gas increases to be a determined level using the gas booster 1. The pressure the raw material water increases to a determined level, by using the raw material water pump 3. Both the pressurized raw material gas and the pressurized raw material water are cooled by a cooler (not shown), which are then fed into the line mixer 5. The raw material gas and the raw material water fed into the line mixer 5 are vigorously mixed together under the mechanism described before. The raw material gas becomes fine bubbles to enter the raw material water and to mix with the raw material water, thus enhancing the raw material gas to dissolve.

The raw material water having the dissolved raw material gas, (containing undissolved fine bubbles), is fed into the reaction conduit 7, where the mixture is cooled by the chiller 17, and all the fed raw material gas are converted to the gas hydrate.

In order to realize the full amount of the gas hydration, the following conditions are required. That's to say; the pressure value (P) at the outlet of the reactor conduit is higher than the minimum pressure value for generating hydrate (P₀). The temperature in the reactor conduit is lower than the maximum temperature for generating hydrate (T₀). In addition, when the raw material gas, which has been mixed and dissolved by the line mixer 5, becomes to be hydrated at full amount, several factors are determined. That is, the determined several factors are these for making it possible to deprive the full amount of the heat of generation, which means, flow rate of the raw material liquid, pressure of the raw material liquid, flow rate of the raw material gas, pressure of the raw material gas, cooling capacity, length of the reactor conduit and diameter of the reactor conduit.

In another word, for keeping the full amount of hydration, the above-mentioned seven parameters, which are, flow rate of the raw material liquid, pressure of the raw material liquid, flow rate of the raw material gas, pressure of the raw material gas, cooling capacity, length of the reactor conduit and diameter of the reactor conduit, are to be determined. The relation between the respective the parameters and the hydrate amount is described below.

First of all, the amount of the raw material water and the generated hydrate amount are explained, in case that the full cooling capacity exists, as follows.

In case that the water rate is bigger than the presumed rate of the water which are determined by the number of hydration (noted below), the flow rate of the raw material water has, basically, no relation with the hydrate amount.

The number of hydrate (composition ratio between water and gas: ratio between water molecule and gas molecule in the hydrate) is, theoretically, 5.75 (water molecule 5.75 mol. in comparison to gas molecule 1 mol.), in case of methane hydrate. However, in the actual procedure, the gas molecule is not always entered every basket (cage), which has been formed by the water molecule. So, the number of hydrate is bigger than the 5.75. (That's to say, the water molecule 5.75 mol or more, to the gas molecule 1 mol.)

In case that the raw material water rate is less than the determined one based on the number of hydrate, the hydrate amount is proportional to one of the raw material water rate. In this case, when the generation has been completed, the gas hydrate and the solid hydrate remain mutually.

Strictly speaking, it is presumed that the generation amount changes, influenced by the change of thermal conductivity ratio (the change of cooling efficiency rate) inside piping, being associated with the change of the raw material water rate (the change of the flow rate in the reaction conduit.)

The relation between the raw material gas rate and the generated hydrate amount is, also, similar to the raw material water rate. That is, as long as the full sufficient cooling capacity exists, the gas rate has no relation with the generated hydrate amount, in case that the gas rate is bigger than the determined one based on the number of hydration.

On the contrary, in case that the gas rate is smaller than the determined gas amount by the number of hydration, the generated hydration amount is proportional to the gas flow rate. In this case, when the generation has completed, the raw material water and the solid hydrate remain.

In Embodiment 2, FIG. 7 shows that pump 19 is provided for returning the non reacted raw material water to line mixer, wherein the non reacted raw material water has been separated by the separator. In this case, it is assumed that the raw material water rate is bigger than the determined one based on the number of hydrate. And it is assumed that the hydrate is generated by supplying, less rate of the raw material gas.

As the next explanation, the relation between the raw material water rate, the pressure and the temperature of the raw material gas and the amount of the generated hydrate are described below.

Within a range of hydrate generation, the higher pressure and the lower temperature invite the easier way to generate the hydrate. Therefore, in case that there exists a sufficient cooling capacity (the deprived heat amount per hour), the higher pressure and the lower temperature invite the faster rate to generate the hydrate. When the cooling capacity rate has a limitation value, the generation rate is influenced by the cooling capacity.

When the raw material gas and the raw material water are mixed and dissolved each other, the pressure of both substances is equal, except when considering from the minute point of view.

Furthermore, on the primary stage of mixing, there occurs, from time to time, the difference between the temperature of the raw material gas and that of the raw material water. However, both of the temperature becomes the equal value, while the raw material gas and that of the raw material water flow in the reaction conduit.

Additionally, there describes the relation between the cooling capacity and the amount of the generated hydrate.

In case that the raw material gas is methane, the amount of heat value (heat of generation) is as follow, which means per one mol of methane.
approximately 14.5 kcal/mol(at 0° C.)
approximately 17 kcal/mol (at 10° C.)

If there exists a sufficient gas defusing into the raw material water or gas dissolving into the raw material water, the amount of the generated hydrate is proportional to the cooled heat value (the deprived heat value). Therefore, even when there exist a sufficient gas defusing into the raw material water and a sufficient gas dissolving gas into the raw material water, the temperature of the raw material water, in which the raw material gas has been dissolved, has a possibility to rise up. The possibility is realized in accordance with hydrate generation, by the reason of the insufficient cooling capacity. In this situation, when the temperature reaches the hydrate generation maximum temperature (the temperature is higher in accordance with the higher pressure), the generation stops. And, at that time, if there exists an unreacted raw material gas, the unreacted raw material gas remains as a gaseous state, which is the dissolved gas in the raw material water or the bubble form in the raw material water. In another word, a sufficient cooling capacity makes it possible to keep the adequate temperature within a range of generation, while all rate of the raw material gas are hydrated.

Supplementary speaking, in case of the excessive cooling capacity, the liquid temperature in the reaction conduit decreases, although the hydration goes on. So, sometimes, it is likely to be frozen. By that reason, a big deal of cooling capacity is not always suitable.

The cooling capacity is determined by the thermal conductivity capacity, which is estimated by the chiller capacity, the specification of the reaction conduit (the length of reaction conduit, diameter, thickness, material characteristics, etc.), taking into consideration the temperature difference between the refrigerant and the liquid in the reaction conduit.

Finally, there describes the relation between the length of reaction conduit/the diameter of reaction conduit and the amount of the generated hydrate.)

Generally speaking, the length of-the reaction conduit and the diameter of the reaction conduit are determined for making use of the cooling capacity of the chiller, sufficiently. Therefore, the length of the reaction conduit and the diameter of the reaction conduit are not concerned with the hydrate amount, respectively and individually. By way of the parameter, which means the cooling capacity, they relate with the hydrate generation. The detailed explanation is as follows.

Concerning the relation between the length of the reaction conduit and the diameter of, the reaction conduit, the longer the length of the reaction conduit is, the bigger the cooling capacity is, based on the similar condition about other factors. The relation between the diameter of reaction conduit and the cooling capacity is more complex a bit. The shorter the diameter of the reaction conduit is, the faster the flow speed in the reaction conduit is. By that reason, the thermal conductivity ratio in the reaction conduit becomes higher. But, in this case, the surface area of the reaction conduit reduces. From the above-mentioned point of view, the relation is that the balance of both factors determines whether the cooling capacity is increasing or decreasing.

Generally, in case of the heat exchanger, the diameter of the reaction conduit is determined to be shorter, in order to increase the thermal conductivity ratio inside the reaction conduit. Simultaneously taking the reduction of the surface area into consideration, the optimal specification is determined to cover the area reduction by increasing the length of the reaction conduit or increasing of the number. The optimal specification invites the advantageous investment cost reduction.

The relation between the produced amount of hydrate and seven parameters are described as mentioned above. Based on the appropriate determined parameters, mechanism of the full amount of hydration is described, as follows.

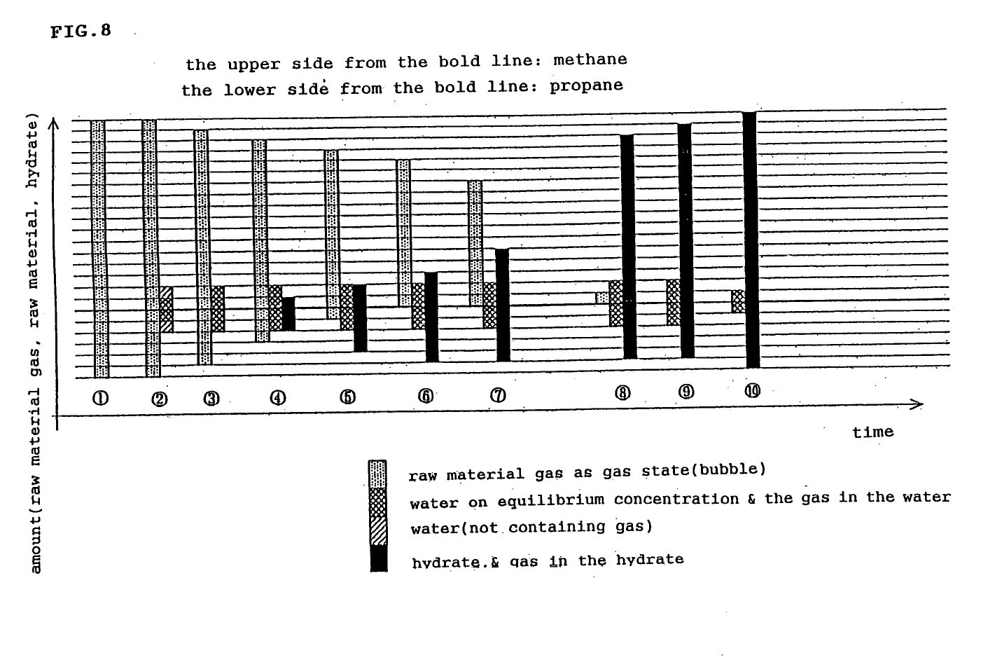

FIG. 8 illustrates the mechanism of total forming of hydrate in the reaction conduit 7, giving a schematic expression of conversion of the raw material gas to hydrate with time, focusing on a certain amount of raw material gas being fed to the reaction conduit 7.

The vertical axis of FIG. 8 is the quantity of raw material gas, raw material water (hereinafter the term “raw material water” means either sole raw material water or raw material water containing dissolved raw material gas), and gas hydrate. The histogram zone above the thick line designates methane, and the zone below the thick line designates propane. The horizontal axis of FIG. 3 is the time, giving emphasized times as \mathring{1} through \mathring{10}. (To clearly identify the corresponding positions of \mathring{1} through \mathring{10}, those indications are given in FIG. 7 at respective points.) For convenience of description, the raw material gas assumes a mixture of methane and propane, with the ratio of methane 17 to propane 16, (refer to \mathring{1}).

In the line mixer 5, the raw material gas, the recycled water (the raw material water containing dissolved mixed gas to an equilibrium concentration), and a make-up water are mixed together, (refer to \mathring{2}). FIG. 3 shows a state that no gas is dissolved immediately after the mixing.

The line mixer 5 makes the raw material gas fine bubbles, which bubbles dissolve in the raw material water, thus bringing the total raw material water to an equilibrium concentration, (refer to \mathring{3}).

When the raw material water reaches an equilibrium concentration of the dissolved raw material gas, the forming of gas hydrate begins because the operating conditions are set so as the pressure P in the reaction conduit 7 to become higher than the minimum pressure P₀ for forming hydrate, and so as the temperature T at each section in the reaction conduit 7 to become lower than the maximum temperature T₀ for forming hydrate. At that moment, although methane and propane are dissolved in the raw material water, propane is more likely converted to hydrate so that the formed gas hydrate contains larger percentage of propane than the percentage thereof in the raw material gas, (refer to \mathring{4}: in the FIG., the histogram indicating the gas hydrate quantity becomes one scale above the thick line and two scales below thereof).

The forming of gas hydrate generates reaction heat. Nevertheless, the temperatures in the reaction conduit 7 are maintained to lower level than the maximum temperature T₀ for forming hydrate by removing the heat equivalent to the generated heat through the cooling by chiller 17. Excessive cooling results in solidifying the raw material water to hinder the smooth flow inside the reaction conduit 7. Accordingly, the cooling capacity of the chiller 17 is set so as the raw material water not to become below the solidification point thereof.

Once the gas hydrate is formed, the quantity of raw material water decreases. However, to avoid the FIG. becomes complex, FIG. 8 gives an expression that the quantity of raw material water does not vary during the period of from \mathring{4} to \mathring{9}.

When the gas hydrate is formed, the dissolved gas concentration decreases, and then the raw material gas further dissolves in the raw material water until the equilibrium concentration is again established, thus further forming the gas hydrate containing larger percentage of propane, (refer to \mathring{5}, \mathring{6}). The formed gas hydrate flows through the reaction conduit 7 together with the raw material water.

All the propane dissolved in the raw material water in \mathring{6}. After the time \mathring{6}, therefore only methane dissolves in the raw material water to begin the forming of gas hydrate containing larger percentage of methane than that of the raw material gas, (refer to \mathring{7}), and similar reaction continues, (refer to \mathring{8}, \mathring{9}).

At the exit of the reaction conduit 7, all the raw material gas is converted to hydrate, (refer to \mathring{10}), which hydrate is then sent to the separator 9 together with the raw material water.

After the beginning of reaction in the reaction conduit 7, the separator 9 receives both the gas hydrate which contains large percentage of propane, formed in the early period, and the gas hydrate which contains large percentage of methane, formed in the later period. Nevertheless, since all the raw material gas is converted to hydrate, the formed hydrate as a total has the same composition as that of the raw material gas.

At the point \mathring{10}, the expression is given in a state of summing up the reduction in the raw material water quantity through the reactions over a period of from \mathring{4} to \mathring{10}. At the point \mathring{10}, the raw material water at an equilibrium concentration is left, which raw material water is recycled to the line mixer 5 by the raw material water pump 19.

The formed gas hydrate is taken out from the separator 9, and is fed to the succeeding process (S5 and following processes in FIG. 11).

In the separator 9, a level gauge 21 detects the water level, and the water level in the separator 9 is controlled to be the equal or higher than the predetermined level by controlling the valve 12d. The control is done to provide the raw material water with sealing effect so as the raw material water not to enter the recycle line of the raw material water. The pressure of the raw material water, which is not needed in the water seal, increases to a determined level by the raw material water pump 19, as described above, and is fed into the line mixer 5.

As described above, according to the Embodiment, the raw material gas is continuously dissolved in the raw material water by the line mixer 5, and all the fed raw material gas is converted to hydrate using the pipe-shape reaction conduit 7, thus the formed gas hydrate has the same composition with that of the fed raw material gas.

According to the Embodiment, the reaction between the raw material water and the raw material gas is carried out while they move through the reaction conduit. As a result, all the reactants, (formed gas hydrate and the raw material water), are once sent to the separator 9, which eliminates the means to separate only the formed gas hydrate, thus simplified facility is attained.

The above-given description deals with the case that all the fed raw material gas is converted to hydrate at the exit of reaction conduit 7. If, however, not all the raw material gas is converted to hydrate in the reaction conduit 7 depending on operating conditions, the following-given means may be applied.

If not all the raw material gas is converted to hydrate in the reaction conduit 7, unreacted raw material gas is fed into the separator 9. In that case, the internal pressure of the separator 9 increases. Therefore, detection of the increase in internal pressure of the separator 9 notifies whether or not the total amount of raw material gas is converted to hydrate in the reaction conduit 7.

Consequently, the pressure detector 10 mounted in the separator 9 detects the pressure increase in the separator 9. When the detected pressure exceeds a predetermined level, it is judged that the raw material gas enters the separator without fully converted to hydrate. The opening of the valve 12a is then decreased to reduce the supply rate.

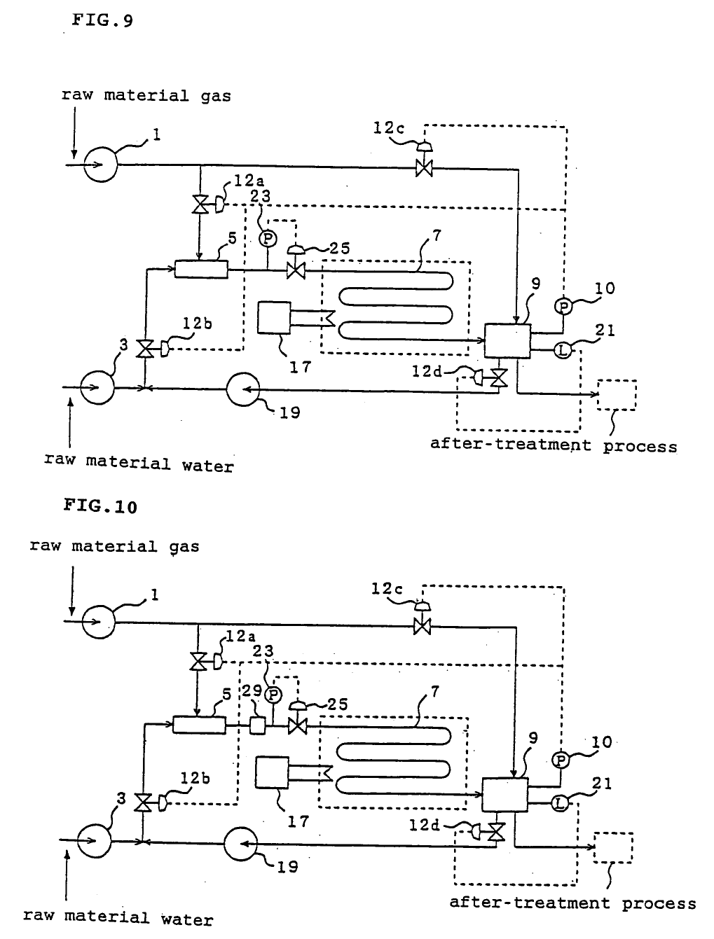

The surplus raw material gas fed to the separator 9 is converted to hydrate in the separator 9, and the internal pressure of the separator 9 can be lowered to the specified level. If sole conversion to-hydrate within the separator 9 cannot decrease the internal pressure of the separator 9, a recycle piping from the separator 9 to the line mixer 5 may be applied to return the surplus raw material gas. The means is also applicable to FIG. 9 and FIG. 10.

The Embodiment is not applied to pressure regulating means between the line mixer 5 and the reaction conduit 7.

As shown in FIG. 9, however, a pressure detector 23 and a pressure regulating valve 25 may be located between the line mixer 5 and the reaction conduit 7.

Adjusting the pressure regulating valve 25 enables the pressure at the line mixer 5 side to increase, thus enhancing the raw material gas in the raw material water to dissolve into the line mixer 5.

In order to further enhance the raw material gas to dissolve into the raw material water, a holding section 29 as the flow speed regulating means for decreasing the flow speed of the fluid flowing through the line may be located at downstream side of the line mixer 5, as shown in FIG. 5. With the holding section 29, the raw material gas, which became fine bubbles in the line mixer 5, acquires a time for dissolving in the raw material water, which enhances the raw material gas to dissolve.

An example of the holding section 29 is a tank having a certain volume.

Further examples of line mixer include the one such as Venturi type, which has a thinning portion in the middle of the cylinder to generate negative pressure for sucking and mixing the raw material gas, or the one in cone or truncated cone shape to create swirling flow therein to conduct mixing of gas and liquid. An example of the latter type mixer is a swirling type fine bubble generator disclosed in Japanese Patent Laid-Open No. 2000-447. In short, the line mixer according to the present invention broadly includes the unit which is located in the line and which is able to mix gas with liquid in continuous mode.

The Embodiment adopts a single or plurality of curved pipes as an example of the reaction conduit 7. The reaction conduit 7, however, may be structured by branched plurality of straight pipes.

The above-given Embodiment does not specify the kind of raw material water. Examples of the raw material water include plain water, seawater, and antifreeze liquid. Instead of the raw material water, liquid host material or host material solution may be applied. In such a case, the formed material is not called the gas hydrate, but called the gas clathrate.

### Embodiment 3

The method for producing gas hydrate according to Embodiment 3 is conducted through the reaction between a raw material water and a raw material gas. The method has the steps of: mixing the raw material water with the raw material gas and dissolving the raw material gas in the raw material water in the route for producing the gas hydrate; and cooling the mixed and dissolved mixture, while the mixture flows through a reaction conduit to form the gas hydrate; sizes of the formed gas hydrate being changed, resulting from changing one or both of the flow speed of the raw material water, which flows through the reaction conduit in the gas hydrate forming step, and the flow rate of the raw material gas, which is fed into the reaction conduit.

Furthermore, the method for producing gas hydrate through the reaction between a raw material water and a raw material gas has the steps of: mixing the raw material water with the raw material gas and dissolving the raw material gas in the raw material water in the route for producing the gas hydrate; and cooling the mixed and dissolved mixture, while the mixture flows through a plurality of reaction conduits to form the gas hydrate; sizes of the formed gas hydrate formed in the respective reaction conduits in the gas hydrate forming step being changed, resulting from changing one or both of the flow speed of the raw material water, which flows through each of a plurality of the reaction conduits and the flow rate of the raw material gas, which is fed into the respective reaction conduits.

Furthermore, an apparatus for producing gas hydrate, in accordance with Embodiment 3, through the reaction between a raw material water and a raw material gas has: a line mixer which mixes the raw material water with the raw material gas and dissolves the raw material gas in the raw material water in the route for producing the gas hydrate; a reaction conduit which cools the mixed and dissolved mixture; and a flow speed controlling means for changing the flow speed of the raw material water, which flows through the reaction conduit.

Furthermore, the apparatus for producing gas hydrate through the reaction between a raw material water and a raw material gas has: a line mixer which mixes the raw material water with the raw material gas and dissolves the raw material gas in the raw material water in the route for producing the gas hydrate; a plurality of reaction conduits which cool the mixed and dissolved mixture; and a flow speed controlling means to change the flow speed of the raw material water flowing through the plurality of reaction conduits; the flow-speed controlling means for controlling the flow speed of the raw material water flowing through the individual reaction conduits, so as the respective flow rate to be different mutually.

Furthermore, the apparatus for producing gas hydrate through the reaction between a raw material water and a raw material gas has: a line mixer which mixes the raw material water with the raw material gas and dissolves the raw material gas in the raw material water in the route for producing the gas hydrate; a gas flow rate regulating means for changing the flow rate of the raw material gas, which is fed into the line mixer; and a reaction conduit, which cools the mixed and dissolved mixture.

Furthermore, the apparatus for producing gas hydrate through the reaction between a raw material water and a raw material gas has: a plurality of dissolving and mixing units, each of the dissolving and mixing units comprises a line mixer, which mixes the raw material water with the raw material gas and dissolves the raw material gas into the raw material water in the route for producing the gas hydrate and a gas flow rate regulating means for adjusting the flow rate of the raw material gas, which is fed to the line mixer; a plurality of reaction conduits, which cool the mixed and dissolved mixture in the respective dissolving and mixing units; the flow speed controlling means for controlling the flow speed of the raw material water in the individual reaction conduits, so as the respective flow rate-to differ mutually.

### Embodiment 3-1

FIG. 15 shows outline of a mode of gas hydrate producing process according to the Embodiment 3-1. The process uses natural gas as the raw material gas. The process for producing gas hydrate is outlined referring to FIG. 15.

The Embodiment 3-1 gives an improvement in the step (S3) that forms slurry gas hydrate from water and natural gas, which improvement is to vary the particle sizes of the formed gas hydrate. The detail of the improvement is described as follows.

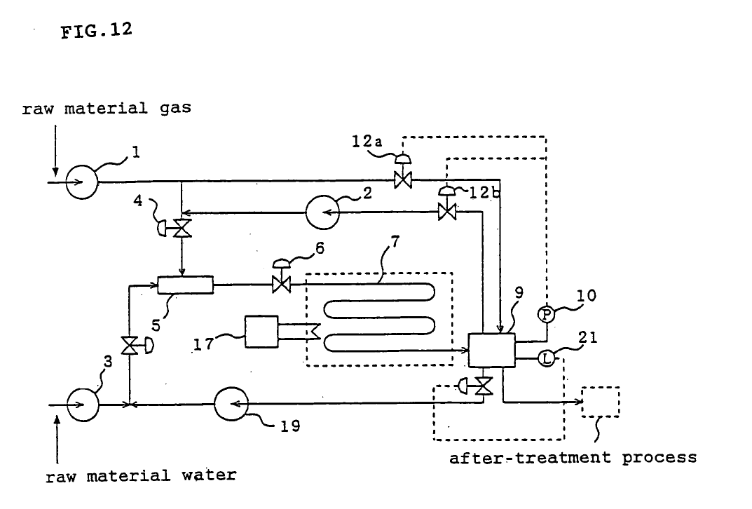

FIG. 12 shows a process diagram of the Embodiment 3-1 for carrying out the present invention, showing the main units. The units of the embodiment for carrying out the present invention are described referring to FIG. 12.

The gas hydrate producing apparatus of the Embodiment 3-1 has gas boosters 1, 2 that increase the pressure of raw material gas such as natural gas, raw material water pumps 3, 19 that feed the raw material water, a line mixer 5 that mixes the raw material water with the raw material gas to dissolve the raw material gas in the raw material water, a reaction conduit 7 that cools the mixture formed by the line mixer 5 to form the gas hydrate, and a separator 9 that separates the gas hydrate formed in the reaction conduit 7, unreacted gas, and the raw material water respectively.

These units are connected to each other by pipes shown in the drawing with solid lines with arrow marks. The pipeline that feeds the raw material gas into the line mixer 5 is equipped with a gas flow rate control valve 4 that adjusts the gas flow rate. The gas flow control means 4 and the line mixer 5 have the dissolving and mixing unit according to the present invention.

On the pipeline connecting the line mixer 5 with the reaction conduit 7, a flow speed control valve 6 that adjusts the flow speed of raw material water containing dissolved raw material gas (including fine bubbles of the gas) is located.

A pressure detector 10 is mounted to the separator 9. Based on the signals transmitted from the pressure detector 10, a valve 12a on the pipeline that feeds the raw material gas into the separator 9 and a valve 12b that recycles the gas from the separator 9 to the line mixer 5 are controlled.

Further the detailed description is given below for critical units in the line.

The line mixer 5 in the Embodiment 3-1 has a cylinder 11 of two stage structure giving a large diameter at inlet and a small diameter at exit, which is illustrated in FIG. 2. (cited from page 7 of catalogue of “OHR line mixer” of Seika Corp.) The cylinder 11 has blades 13 called the guide vanes in the large diameter section 11a, and has a plurality of impingers 15 in mushroom shape, which extend from the inner periphery of the cylinder toward the center thereof in the small diameter section 11b at downstream side of the large diameter section 11a.

In such a type of line mixer 5, the raw material water that is fed by the raw material water pump 3 creates a swirling flow caused by the blades 13. The resulted strong centrifugal force pushes the raw material water outward, whose raw material water then collides against the mushroom impingers 15 to receive stronger agitation. The raw material gas is caught by the vigorous agitation of the raw material water to form very fine bubbles. As a result, the raw material water and the raw material gas are mixed together. Consequently, the contact area between the raw material gas and the raw material water ends up in dissolving the raw material gas into the raw material water efficiently.

A curved pipe structures the reaction conduit 7. A chiller 17 cools the surface of the reaction conduit 7. Using the reaction conduit 7 makes it possible to cool from periphery efficiently. As a result, there is no need in cooling directly the gas and the raw material water-by cooling coil or other means. (Such a direct cooling was applied to the conventional method, though.). Therefore, the constitution of the facility of the embodiment becomes simple and compact.

Such a type of reaction conduit 7 realizes to be applied to the process by mixing and by dissolving the raw material gas and the raw material water in the line mixer 5, in advance. And such a application in advance allows the reaction conduit 7 to exclusively concentrate on cooling. That is, according to the conventional method described in the Patent document 1, the mixing, dissolving, and reaction cooling of the raw material gas and the raw material water are conducted in a tank-shape pressure vessel, so the mixing and dissolving action requires a determined volume of a space, which hinders cooling solely from periphery of the reaction tank. On the contrary, according to Embodiment 1, mixing the raw material gas and the raw material water and dissolving ones are done separately from cooling the reaction, so the reaction step makes it possible to concentrate on cooling. As a result, a simple configuration is applied to cool the process, described above.

The separator 9 separates gas hydrate, unreacted gas, and raw material water respectively. The separator 9 to be usable includes a decanter, a cyclone, a centrifugal separator, a belt press, a screw concentrator dehydrator, and a rotary drier.

The process for producing gas hydrates having different particle sizes among them mutually, which uses a facility of Embodiment 1 having the configuration given above is described as follows.

The pressure of raw material gas increases to a determined level by the gas booster 1. The raw material water, also, increases in the pressure to a determined level by the raw material water pump 3. Both the pressurized raw material gas and the pressurized raw material water are fed to the line mixer 5. The gas flow rate control valve 4 controls the pressurized raw material gas. The raw material gas enters the line mixer 5 at a determined flow rate. The raw material gas and the raw material water, which are fed to the line mixer 5, are vigorously mixed together under the mechanism described before. The raw material gas becomes fine bubbles to enter and to mix with the raw material water, thus enhancing the raw material gas to dissolve into the water.

The raw material water that has the dissolved raw material gas, (containing fine bubbles), is controlled to a determined flow speed by the flow speed control valve 6. And the raw material water is fed into the reaction conduit 7, where the mixture is cooled by the chiller 17 to form the gas hydrate. The formed gas hydrate, along with the unreacted gas and the raw material water, flows through the conduit to enter the separator 9. In such a manner, the gas hydrate having a determined particle size is produced to a degree of the constant amount.

The following is the description about the method for producing the gas hydrate having different particle sizes respectively.

In order to form the gas hydrates having different particle sizes respectively, the respective control valves 4, 6 are adjusted. The mechanism is described below, that is, how to vary the respective particle size under the control of adjusting each control valve.

As a premise of the different particle sizes of the hydrate respectively, the mechanism, which is how to form the hydrate in the reaction conduit 7, is described. That is, the line mixer 5 mixes the raw material gas with the raw material water. The raw material gas becomes fine bubbles to dissolve into the raw material water. And the whole amount of the raw material water reaches an equilibrium concentration of the raw material gas.

When the raw material water reaches an equilibrium concentration, the gas hydrate begins to form, because the operating conditions are determined so as the pressure P in the reaction conduit 7 to become higher than the minimum pressure P₀ for forming hydrate, and so as the temperature T at each section in the reaction conduit 7 to become lower than the maximum temperature T₀ for forming hydrate. When the gas hydrate is formed, the reaction heat generates. Nevertheless, the temperatures in the reaction conduit 7 are kept to a lower level than the maximum temperature To for forming the hydrate by deriving the heat, which is equivalent to the generated heat through the cooling by chiller 17. In this case, the excessive cooling ends up in solidifying the raw material water to hinder the smooth flow inside the reaction conduit 7. Accordingly, the cooling capacity of the chiller 17 is determined so as the raw material water not to become equal or less than the solidification point.

Once the gas hydrate is formed, the dissolved gas concentration decreases, and then the raw material gas further dissolves into the raw material water until the equilibrium concentration is again established. When the concentration becomes equal or higher than the equilibrium concentration, the further quantity of the gas hydrate is formed. In such a case, the formed gas hydrate grows up to be coarse particles, compared with the precedent gas hydrate. The formed gas hydrate flows through the reaction conduit 7, and enters the separator 9 together with the raw material water and the unreacted gas (The unreacted gas does not exist in case that the total volume of the gas has converted to the hydrate).

In such a mechanism of to form the gas hydrate, when the flow speed control valve 6 is adjusted to increase the flow speed of the fluid, which flows through the reaction conduit 7, the flow speed of gas hydrate flowing through the reaction conduit 7 increases. Thus, such a mechanism decreases the retention time of the gas hydrate, which is formed at upstream zone of the reaction conduit 7 in the reaction conduit 7. As a result, it spends less time for the formed gas hydrate to grow up to [symbol] be crystals, in the upstream zone of the reaction conduit 7. As a result, the gas hydrate that has a smaller particle size is fed into the separator 9.

Contrarily, in case that the flow speed control valve 6 is adjusted to decrease the flow speed of the fluid, which flows through the reaction conduit 7, it spends longer time for the gas hydrate, which is formed in the upstream zone of the reaction conduit 7, to retain in the reaction conduit 7. By that reason, it spends longer time for the crystal of the gas hydrate, which has already been formed in the upstream zone, to grow up. Thus, the gas hydrate particles having a coarse size of the respective particles are sent to the separator 9.

If the gas flow rate control valve 4 is adjusted to decrease the gas flow rate, the raw material gas that dissolved in the raw material water in the upstream zone of the reaction conduit 7 becomes the hydrate. Even if the mixture flows to the downstream side, the supplied amount of the raw material gas is small so that there becomes no raw material gas to dissolve into the raw material water, in the downstream zone.

As a result, without growth of the crystals of the formed gas hydrate, the gas hydrate is fed to the separator 9.

Consequently, the particle size of the formed gas hydrate becomes smaller.

Contrarily, if the gas flow rate control valve 4 is adjusted to increase the gas flow rate, the raw material gas dissolved into the raw material water becomes hydrate in the upstream zone of the reaction conduit 7. And, the raw material gas further dissolves into the raw material water toward the downstream side. Thus, the crystals of the formed gas hydrate grow up to be large, and then enter the separator 9. As a result, the particle size of the formed gas hydrate becomes coarse.

As described above, in order to make the particle size of the formed gas hydrate small, one countermeasure is to increase the flow speed of the fluid flowing through the reaction conduit 7 by adjusting the flow speed control valve 6. The other one is to decrease the gas flow rate by adjusting the flow rate control valve 4. Otherwise, both of them are usable.

On the contrary, in order to make the particle size of the formed gas hydrate large, one countermeasure is to decrease the flow speed of the fluid flowing through the reaction conduit 7 by adjusting the flow speed control valve 6. The other one is to increase the gas flow rate by adjusting the flow rate control valve 4. Otherwise, both of them are usable.

The adjustment of each control valve 4 or 6 may be done at regular intervals by manual operation, or may be done by automated control means actuated at a predetermined interval.

As described above, the adjustment of each control valve 4 or 6 makes it possible to feed the gas hydrate having the respective different particle sizes into the separator 9. Here, the gas hydrate, the unreacted gas, and the raw material water are separated respectively. The separated raw material water is recycled to the line mixer 5 by the pump 19. The pressure of the unreacted raw material gas increases to-a determined level by the gas booster 2 to feed to the line mixer 5.

The formed gas hydrate is taken out from the separator 9, and is fed to the succeeding process (S5 and following processes in FIG. 15). Since the gas hydrates having different particle sizes respectively are mixed together, the volumetric packing efficiency after dewatered and formed increases. Therefore, the bulk density becomes high, resulting in reducing the transportation cost.

In the separator 9, a level gauge 21 detects the water level. And the water level in the separator 9 is controlled to the equal or higher determined level, in order to obtain the raw material water water-seal effect so as the gas not to enter the raw material water recycle line. The pressure of the raw material water that is not required for the water seal increases to a degree of a determined level by the raw material water pump 19, and is fed to the line mixer 5.

As described above, according to this Embodiment, the various methods make it possible to form continuously the gas hydrates having the respective different particle sizes. These are to say, the gas flow rate control valve 4 and the flow speed control valve 6 are applied to the process, and each of the control valves 4, 6 is adjusted at a regular interval.

According to this Embodiment, the reaction between the raw material gas and the raw material water is carried out, while forcing the raw material gas and the raw material water to move through the conduit. So, in this the gas hydrate-forming step, all of the materials is fed to the separator 9. (All of the materials are the formed gas hydrate, the unreacted gas, and the raw material water) That is, there is no need in taking out merely the formed gas hydrate, although it is necessary in the case of conventional method.) And the configuration of the facility becomes simple.

As described above, the line mixer 5 in cylindrical shape continuously does dissolving the raw material gas in raw material water, thus the dissolution is conducted in a space-saving and efficient manner.

Furthermore, dissolving the raw material gas into the raw material water in the line mixer 5, which is arranged separately from the reactor, enables the pipe-shape reaction conduit 7 to be usable for the process, without taking into consideration a large-sized reactor as the conventional method. This embodiment makes it possible to apply simple and compact cooling means that cools the peripheral surface of the conduit to the process.

In addition, both dissolving the raw material gas in the line mixer 5 and forming the gas hydrate in the reaction conduit 7 are done in the continuous mode, so that this embodiment brings up drastically the efficient increase in producing the gas hydrate.

### Embodiment 3-2

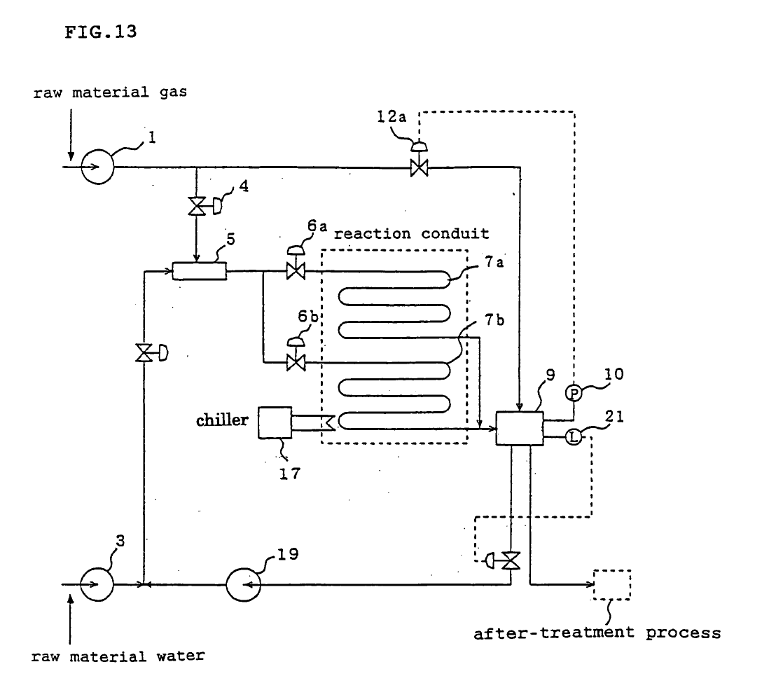

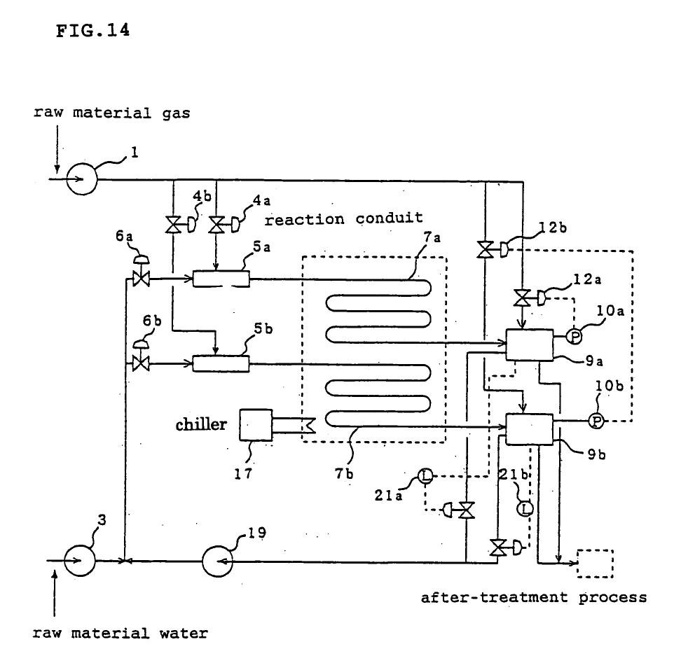

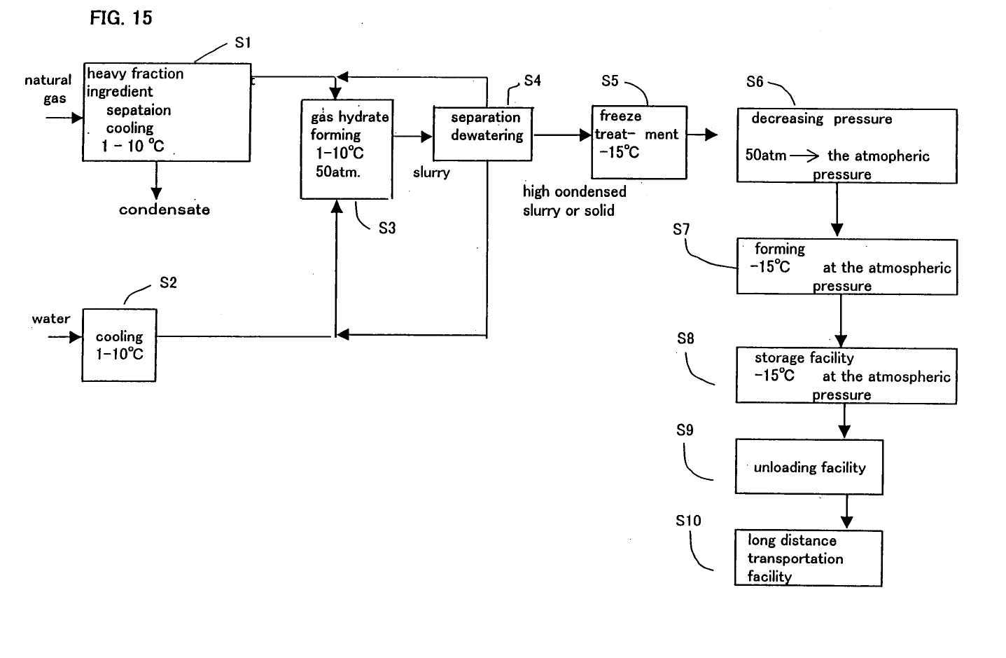

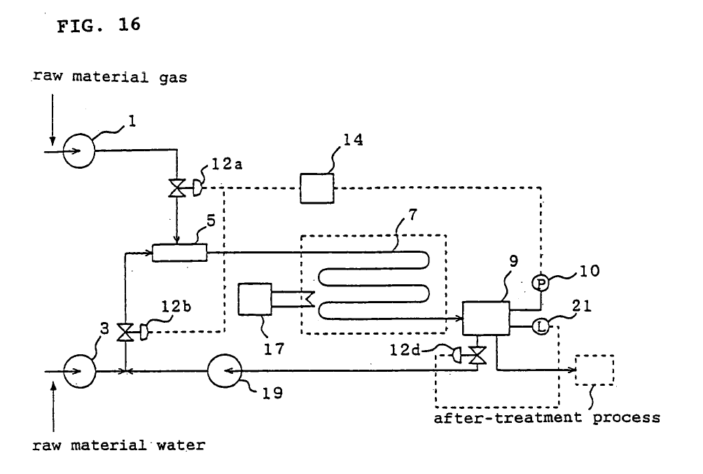

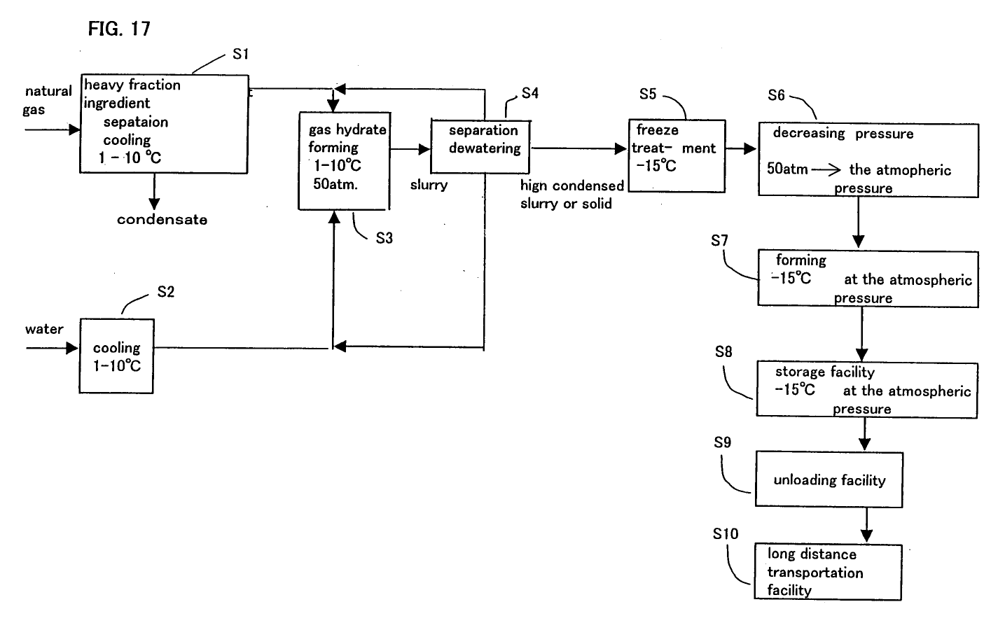

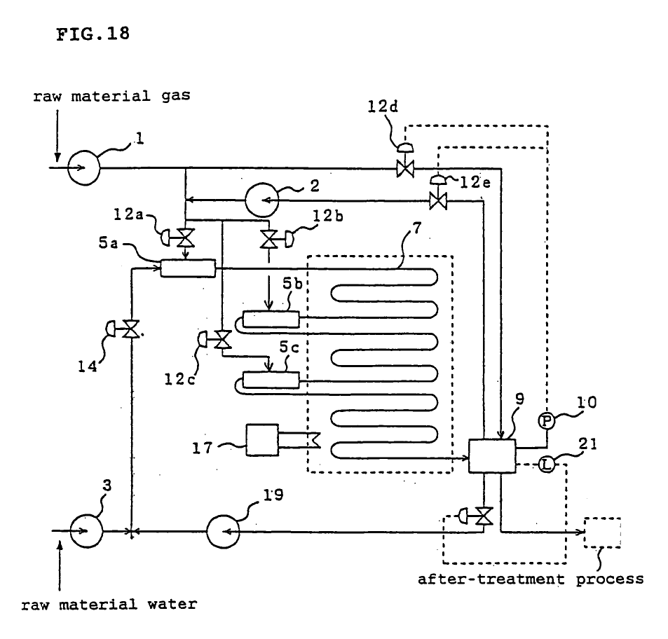

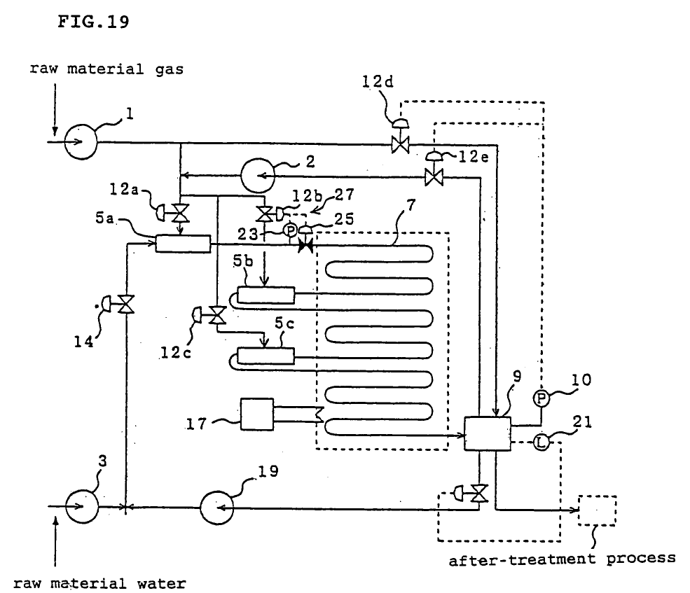

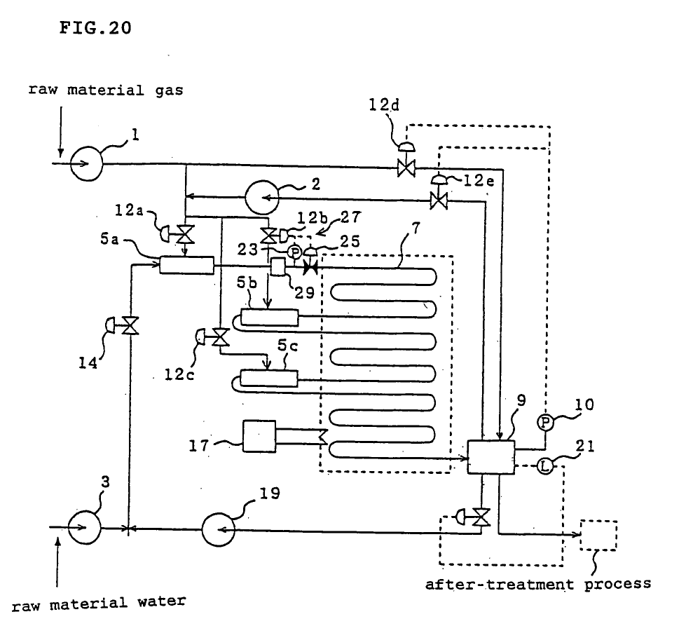

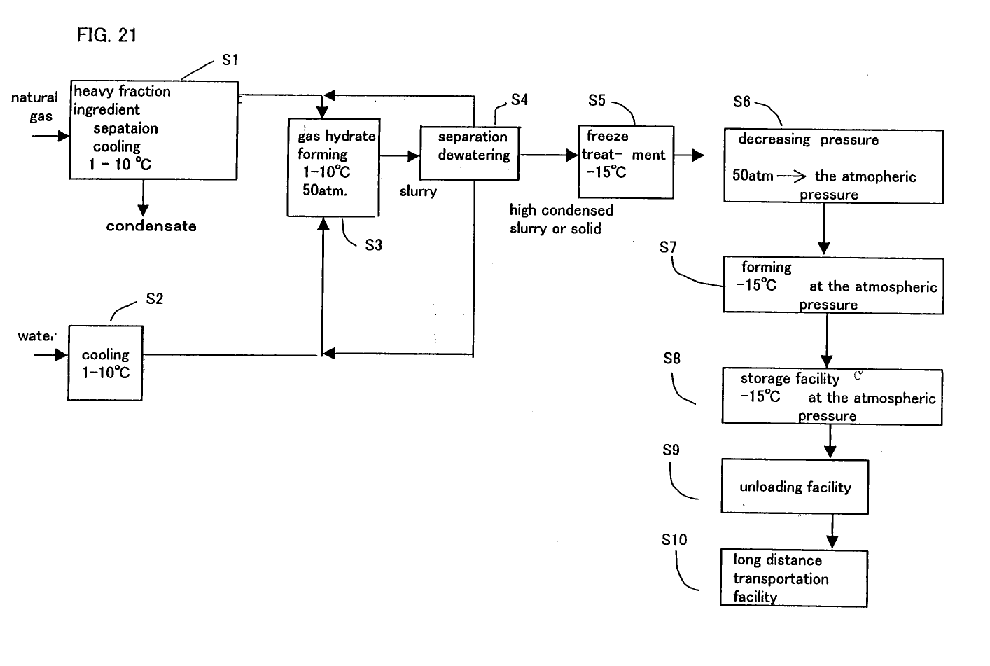

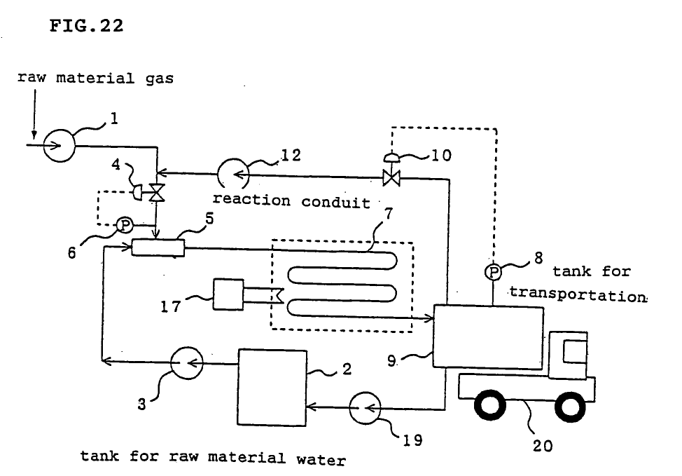

FIG. 13 shows a flow diagram of the Embodiment 3-2 according to the present invention, giving main units. For the same section with that in FIG. 12, the same reference symbol is given.

According to the Embodiment 3-2, two reaction conduits 7a, 7b are located, and flow speed control valves 6a, 6b are mounted to the inlet of the respective reaction conduits.

According to the Embodiment 3-2 having the configuration described above, adjusting flow speed control valves 6a, 6b makes it possible to change the flow speed of fluid flowing through the respective reaction conduits 7a, 7b. This enables the gas hydrates to generate simultaneously the respective different particle sizes among them mutually. And, such gas hydrate, which have different particle sizes, are fed into the separator 9.

In the above-given example, the following devices are applied to the process. That is, the flow speed control valves 6a, 6b as a means for changing the flow speed of fluid this Embodiment do not limit to this configuration. In the exemplary embodiment, it may be a case, the respective pipes have the respective pipe-size (diameter-size), as the reaction conduits 7a, 7b.

### Embodiment 3-3

FIG. 14 shows a flow diagram of the Embodiment 3-3 according to the Embodiment, giving main units. For the same section with that in FIG. 12 or FIG. 13, the same reference symbol is given.

According to the Embodiment, two line mixers 5a, 5b, two reaction conduits 7a, 7b, and two separators 9a, 9b are located, and the gas flow rate control valves 4a, 4b, and the flow speed control valves 6a, 6b are mounted to the inlet of the respective line mixers 5a, 5b.

According to the Embodiment having such a configuration as described above, adjusting the gas flow rate control valves 4a, 4b and the flow speed control valves 6a, 6b makes it possible to change the flow speed of fluid and the flow rate of gas flowing through respective reaction conduits 7a, 7b. This enables the gas hydrates to generate_simultaneously the respective different particle sizes among them mutually. And, such gas hydrate, which have different particle sizes, are fed into the separator 9a and 9b.

The gas hydrates fed to respective separators 9a, 9b are mixed together before the forming step (S7) in FIG. 15.

According to the Embodiment, the gas flow rate control valves 4a, 4b and the flow speed control valves 6a, 6b are mounted to vary both the gas flow rate and the fluid (raw material water) flow speed, so a fine control of particle size is available.

The above-given description on Embodiments 3-1 to 3-3 does not express explicitly the details of the temperature and the pressure in the individual steps. As one of the exemplary example, there is FIG. 15. However, the value of the temperature and that of the pressure at each step are optimally selected.

In the above-given Embodiments 1 to 3, there has been described the natural gas, which consists mainly of methane gas as the raw material gas. But, as other examples of the raw material gas, there are followed up, which are, ethane, propane, butane, krypton, xenon, and carbon dioxide.

Furthermore, as one example of the line mixer, it may be a case, there is a line mixer such as Venturi type. The line mixer such as Venturi type has a thinning portion in the middle of the cylinder to generate the negative pressure for sucking and mixing the raw material gas. Otherwise, there is a line mixer such as in cone shape or in truncated cone shape in order to create swirling flow to conduct mixing of gas and liquid. As an example of the latter type mixer, a swirling type fine bubble generator is followed up, such as disclosed in Japanese Patent Laid-Open No. 2000-447. In short, the line mixer according to this Embodiment is broadly defined as the unit that is located on the way of the line and that enables the process to mix gas with liquid continuously.

According to the above-given Embodiment, as one of the examples, the reaction conduit 7 is shown, which has a single or a plurality of curbed pipes. However, it may be a case, the reaction conduit has three or more of curbed pipes. In case that the three or more of the curbed pipes are applied to the process, it makes it possible to form the gas hydrate, which has the furthermore different particle sizes respectively, and simultaneously.

In the above-mentioned Embodiments, the sorts of the raw material water are not expressed explicitly. Following the example, the raw material water includes plain water, seawater, and antifreeze liquid. Additionally, instead of the raw material water, liquid host material or host material solution may be applied to the process. In such a case, it goes without saying that the formed substance (material) is not named for the gas hydrate but is named for the gas clathrate.

### Embodiment 4

The method for producing gas clathrate according to Embodiment 4 is a method containing the steps of: mixing the raw material liquid with the raw material gas and dissolving the raw material gas in the raw material liquid in the route for producing the gas clathrate; forming the gas clathrate by cooling the mixed and dissolved mixture while the mixture flows through a reaction conduit; separating the formed gas clathrate in a separator, which is connected with the reaction conduit; detecting pressure in the separator; and regulating the pressure in the separator by adjusting one or both of the flow rate of the gas, which is fed to the mixing and dissolving step, and the flow speed of raw material liquid in the step of forming gas clathrate, based on the pressure detected in the pressure detecting step.

The apparatus for producing gas clathrate according to Embodiment 4 is an apparatus contains: a gas flow rate regulating means for adjusting the flow rate of-the feeding raw material gas; a raw material liquid flow speed regulating means for adjusting the flow speed of the feeding raw material liquid; a line mixer for mixing the raw material liquid with the raw material gas and for dissolving the raw material gas in the raw material liquid in a route, in order to produce the gas clathrate; a reaction conduit for cooling the raw material liquid mixed with the raw material gas and for dissolving the raw material gas while the mixture flows in the reaction conduit; a separator connected to the reaction conduit, whose separator separates the formed gas clathrate; a pressure detecting means for detecting the pressure of the separator; and a control means for adjusting one or both of the gas flow rate in the gas flow rate regulating means and the raw material liquid flow speed in the raw material liquid flow speed regulating means, based on the pressure detected in the pressure detecting step.

The line mixer is characterized in the devices that generates fine bubbles of the raw material gas.

As one of the exemplary embodiment of gas clathrate, gas hydrate is described below.

FIG. 17 shows an outline of Embodiment 4, concerning gas hydrate producing process. The process uses natural gas as the raw material gas.

The Embodiment according to the Embodiment gives an improvement in the step (S3) that forms slurry gas hydrate from water and natural gas, and in the step (S4) that separates and dewaters the gas hydrate, thus actualizing an efficient hydrate production and simplified apparatus. The improvement is described in detail, as follows.

FIG. 16 shows a process diagram for carrying out the Embodiment 4, giving main units. The units of Embodiment 4 are described referring to FIG. 16.

The gas hydrate producing apparatus of Embodiment 4 has a gas booster 1, which increases the pressure of raw material gas such as natural gas, raw material water pumps 3, 19 that feed the raw material water (“raw material water” referred in this specification is defined as either sole raw material water or the raw material water containing dissolved raw material gas) a line mixer 5 that mixes the raw material water with the raw material gas in order to dissolve the raw material gas in the raw material water, a reaction conduit 7 that cools the mixture formed by the line mixer 5 in order to form the gas clathrate while the mixture is forced to flow in the reaction conduit, and a separator 9 that separates the gas clathrate formed in the reaction conduit 7, the unreacted gas, and the raw material water respectively.

Such units are connected to each other by pipes given in the drawing with solid lines with arrow marks. The pipeline that feeds the raw material gas to the line mixer 5 is equipped with a gas flow rate control valve 12a that adjusts the gas flow rate. The pipeline connecting the raw material water pumps 3, 19 with the line mixer 5 is equipped with a flow speed control valve 12b that adjusts the flow speed of the raw material water. The separator 9 is equipped with a pressure detector 10 that detects the pressure in the separator 9. Based on the signals transmitted from the pressure detector 10, a control means 14 controls the gas flow rate control valve 12a and the flow speed control valve 12b.

Further detailed description is given below for critical units in the line.

The line mixer 5 in the Embodiment according has a cylinder 11 of two-stage structure giving a large diameter at the inlet and a small diameter at the exit. This is illustrated in FIG. 2. The cylinder 11 has blades 13 called the guide vanes in the large diameter section 11a, and has plurality of impingers 15 in mushroom shape extending from the inner periphery of the cylinder toward the center thereof in the small diameter section 11b at downstream side of the large diameter section 11a.

In such a type of line mixer 5, the raw material water that is fed by the raw material water pump 3 creates a swirling flow caused by the blades 13. The resulted strong centrifugal force pushes the raw material water outward, whose raw material water then collides against the mushroom impingers 15 to receive stronger agitation. The raw material gas is caught by the vigorous agitation of the raw material water to form very fine bubbles, thus the raw material water and the raw material gas are mixed together. As a result, the contact area between the raw material gas and the raw material water increases in dissolving efficiently the raw material gas into the raw material water.

A single or a plurality of curved pipes structures the reaction conduit 7. A chiller 17 cools the surface of the reaction conduit 7. The reaction conduit 7 unables the exemplary embodiment to cool the process from periphery. As a result, there is no need in cooling directly the gas and the raw material water by cooling coil or by other means. (The direct cooling is applied to the conventional method, though.) Thus, the configuration of the facility becomes simple and compact.

In advance, such a type of reaction conduit 7 realizes to be applied to the mixing and the dissolving of the raw material gas and the raw material water in the line mixer 5. And this application in advance allows the reaction conduit 7 to exclusively concentrate on cooling. That is, according to the conventional method described in the Patent document 1, mixing, dissolving, and cooling the reaction of the raw material gas and the raw material water are conducted in a tank-shape clathrate-forming vessel, so the mixing and dissolving action requires a determined volume of a space, which hinders the cooling solely from periphery of the vessel. On the contrary, according to the Embodiment, mixing and dissolving the raw material gas and the raw material water are separated from cooling the reaction, so the reaction step makes it possible to concentrate on cooling. As a result, cooling by the simple configuration described above applied to the process.

The separator 9 separates gas clathrate, unreacted gas, and raw material water from each other. The applied separator 9 includes a decanter, a cyclone, a centrifugal separator, a belt press, a screw concentrator dehydrator, and a rotary drier.

The process for producing gas clathrate by using a facility of the Embodiment having the configuration given above is described as follows.

The pressure of raw material gas increases to a determined level by using the gas booster 1. The pressurized raw material gas is fed into the line mixer 5 via the gas flow rate control valve 12a. The pressure of the raw material water also increases to a determined level by the raw material water pump 3. The pressurized raw material water is also fed into the line mixer 5 via the flow speed control valve 12b. At the beginning of the operation, the gas flow rate control valve 12a and the flow speed control valve 12b are adjusted to the maximum value respectively. The raw material gas and the raw material water fed to the line mixer 5 are vigorously mixed together under the control of the mechanism described before. The raw material gas becomes fine bubbles to enter the raw material water and to mix with the raw material water, thus enhancing the raw material gas to dissolve into the raw material water.

The raw material water that includes the dissolved raw material gas, (containing un-dissolved fine bubbles), is fed to the reaction conduit 7, where the mixture is cooled by the chiller 17, then is fed to the separator 9. Since the pressure in the reaction conduit 7 does not reach the clathrate-forming pressure at the beginning of the operation, no clathrate is formed, and the undissolved raw material gas is fed to the separator 9. As a result, the pressure in the separator 9 increases. After a determined time has passed since the operation started, the internal pressure of the separator 9 begins to increase. When the pressure in the reaction conduit 7 connecting with the separator 9 increases to reach the clathrate-forming pressure, the gas clathrate forming begins in the reaction conduit 7. Thus the formed gas clathrate flows through the conduit along with the unreacted gas and the raw material water, and enters the separator 9.

Once the unreacted gas enters the separator 9, the internal pressure of the separator 9 increases. If the internal pressure of the separator 9 exceeds a predetermined level, the control means 14 controls one or both of the gas flow rate control valve 12a and the raw material water flow speed control valve 12b to adjust the pressure in the separator 9 and the pressure in the reaction conduit 7.

Adjusting each of the control valves 12a, 12b, as described above controls the pressure in the separator 9. The mechanism of varying the pressure in the separator 9 by adjusting each control valve is described below.

As a premise of the above-mentioned way, the mechanism of forming clathrate in the reaction conduit 7 is described as follows.

That is, the line mixer 5 mixes the raw material gas with the raw material water. The raw material gas becomes fine bubbles to dissolve in the raw material water, and the whole amount of the raw material water reaches an equilibrium concentration of the raw material gas.

When the raw material water reaches an equilibrium concentration, clathrate begins to be formed. In case that the operational conditions are determined so as the pressure P in the reaction conduit 7 to become higher than the minimum pressure P₀ for forming clathrate, and so as the temperature T at each section in the reaction conduit 7 to become lower than the maximum temperature T₀ for forming clathrate. Forming clathrate generates the corresponding reaction heat. Nevertheless, the temperatures in the reaction conduit 7 are kept to be lower level than the maximum temperature T₀ for forming clathrate by deriving the heat, which is equivalent to the generated heat through the cooling by chiller 17. Excessive cooling results in solidifying the raw material water to hinder the smooth flow inside the reaction conduit 7. Accordingly, the cooling capacity of the chiller 17 is determined so as the raw material water not to become the equal or lower than the solidification point.

Once the clathrate is formed, the dissolved gas concentration decreases, and then the raw material gas further dissolves into the raw material water until the equilibrium concentration is again established. When the concentration becomes the equal or higher than the equilibrium concentration, further quantity of clathrate is formed. The formed clathrate flows through the reaction conduit 7, and enters the separator 9 together with the raw material water and the unreacted gas (the unreacted gas don't exist when the total volume of the gas has converted to clathrate).

In such a mechanism of forming gas clathrate, when the gas flow rate decreases by adjusting the gas flow rate control valve 12a, the ratio of forming of clathrate within the reaction conduit 7 to the quantity of the supplied and fed gas increases, thus the quantity of the unreacted gas fed into the separator 9 decreases. When the quantity of the fed gas decreases to a determined level or below, the total amount of the raw material gas is converted to clathrate, and no unreacted gas is fed into the separator 9. In such a manner, the quantity of the unreacted gas fed into the separator 9 is adjusted by adjusting the gas flow rate control valve 12a and by reducing the gas flow rate.

On the other hand, since the inside of the separator 9 exists also under an environment to form clathrate, the unreacted gas in the separator 9 is dissolved and is converted to clathrate, so the pressure in the separator 9 tends to decrease.

As a result, when the quantity of the unreacted gas fed into the separator 9 decreases or becomes to zero, the reduction of the unreacted gas quantity in the separator 9 resulted from forming the clathrate becomes predominant, which results in decreasing the pressure in the separator 9.

Contrarily, when the gas flow rate increases by adjusting the gas flow rate control valve 12a, the ratio of forming of clathrate within the reaction conduit 7 to the quantity of fed gas decreases. When the quantity of the fed gas increases to a determined level or above, all of the raw material gas is not always converted to clathrate in the reaction conduit 7 and the unreacted gas is fed into the separator 9. In such a manner, the quantity of the unreacted gas fed into the separator 9 increases by increasing the gas flow rate by adjusting the gas flow rate control valve 12a, which increases the pressure in the separator 9.

When the flow speed control valve 12b is adjusted to increase the flow speed of the fluid flowing through the reaction conduit 7, it spends less time for the raw material water to retain in the reaction conduit 7, so that the quantity of the raw material gas dissolving into the raw material water decreases and the quantity of the converted clathrate decreases. Thus increasing the quantity of unreacted gas fed to the separator 9 increases. As a result, the pressure in the separator 9 increases.

On the contrary, when the flow speed control valve 12b is adjusted to decrease the flow speed of the fluid flowing through the reaction conduit 7, it spends long for the raw material water in the reaction conduit 7 to retain, so that the quantity of the raw material gas dissolving in the raw material water and the converted clathrate increases. Thus, there decreases the quantity of unreacted gas fed into the separator 9. As a result, it makes possible to stop increasing the pressure of the separator 9 or to decrease the pressure the separator.

As described above, increase in the pressure of the separator 9 is attained by increasing the gas flow rate through the adjustment of the gas flow rate control valve 12a or by increasing the flow speed of fluid flowing through the reaction conduit 7 through the adjustment of the flow speed control valve 12b.

On the contrary, the pressure of the separator 9 decreases by decreasing the gas flow rate through the adjustment of the gas flow rate control valve 12a or by decreasing the flow speed of fluid flowing through the reaction conduit 7 through the adjustment of the flow speed control valve 12b.

As described above, through the adjustment of each of the control valves 12a, 12b, the pressure in the separator 9 is adjusted, and the pressure in the separator 9 is kept to be a level, which stabilizes the formed clathrate. As a result, the pressure in the reaction conduit 7 is also kept to be an optimum level for forming the clathrate.

In the separator 9, the gas clathrate, the unreacted gas, and the raw material water are separated from each other. The separated raw material water is recycled to the line mixer 5 via the pump 19.

On the other hand, the formed gas clathrate is taken out from the separator 9, and is fed to the succeeding process (S5 in FIG. 17 and following processes).

In the separator 9, the water level is detected by a level gauge 21, and the water level in the separator 9 is controlled to be at or above the determined level to give the raw material water water-seal effect so as the gas not to enter the raw material water recycle line. The raw material water that is not required for the water seal increases in the pressure to a determined level by the raw material water pump 19, and is fed into the line mixer 5.

As described above, according to the Embodiment, the gas flow rate control valve 12a and the flow speed control valve 12b are adopted, and the pressure detector 10 is mounted to each of the control valves 12a, 12b to conduct control on the basis of the pressure detected by each detector 10. Therefore, a simple unit achieves the pressure control, which simplifies the apparatus.

According to the Embodiment, the reaction between the raw material gas and the raw material water is carried out while making the raw material gas and the raw material water move through the conduit. So, in the step of forming gas clathrate, all of the materials (formed gas clathrate, unreacted gas, and raw material water) are sent to the separator 9. That is, there is no need in taking out only the formed gas clathrate, which is necessary in the case of conventional method, and the configuration of the facility becomes simple.

As described above, the line mixer 5 in cylindrical shape continuously does dissolving the raw material gas in the raw material water, thus the dissolution is conducted in space-saving and efficient manner.

Furthermore, dissolving the raw material gas in the raw material water in the line mixer 5, which is separated from the clathrate-forming vessel, allows the process to use of the pipe-shape reaction conduit 7, instead of the clathrate-forming vessel having a large diameter, which makes it possible to apply the simple and compact cooling means that cool the peripheral surface of the conduit to the process.

In addition, both the dissolution of the raw material gas in the line mixer 5 and the forming of gas clathrate in the reaction conduit 7 is done continuously, so that gas clathrate is produced efficiently in a drastic manner.

The above-given description according to the Embodiment does not express the detail of the temperature and the pressure in the individual steps. As one of the exemplary embodiment, it may be a case, FIG. 17 is given. The temperature and the pressure at the respective steps are selected to be the optimum values, influenced by the various conditions.

The above-given Embodiment describes natural gas consisting mainly of methane gas as the raw material gas. Other applicable examples of the raw material gas include ethane, propane, butane, krypton, xenon, and carbon dioxide.

Further examples of line mixer include a line mixer such as Venturi type, which has a thinning portion in the middle of the cylinder to generate negative pressure for sucking and mixing the raw material gas. Otherwise, the line mixer such as in cone or truncated cone shapes to create swirling flow to conduct mixing of gas and liquid. An example of the latter type mixer is a swirling type fine bubble generator disclosed in Japanese Patent Laid-Open No. 2000-447. In short, the line mixer according to the Embodiment broadly includes the unit which is located in the line and which is able to mix gas with liquid continuously.

According to the above-given Embodiment, the reaction conduit 7 is a single or a plurality of curbed pipes, as an example. However, the reaction conduit 7 may be formed by a plurality of straight pipes.

The above-given Embodiment does not specify the kind of raw material water. Examples of the raw material water include plain water, seawater, and antifreeze liquid. Instead of the raw material water, liquid host material or host material solution may be applied.

### Embodiment 5

The method for producing gas clathrate through the reaction between a raw material liquid and a raw material gas, has the steps of: the first mixing and dissolving step, where the raw material liquid is mixed with the raw material gas and the raw material gas is dissolved in the raw material liquid in the route for producing the gas clathrate; the gas clathrate-forming step, where thus mixed and dissolved mixture is cooled while the mixture flowing through a reaction conduit to form the gas clathrate; the separation step, where the formed gas clathrate is separated in a separator connected with the reaction conduit; a single or a plurality of second mixing and dissolving steps being positioned after the first mixing and dissolving step and before the gas clathrate-forming step, or in the gas clathrate-forming step, to dissolve the raw material gas in the raw material liquid.

The apparatus for producing gas clathrate through the reaction between a raw material liquid and a raw material gas, has: a line mixer which mixes the raw material liquid with the raw material gas and dissolves the raw material gas in the raw material liquid in the route for producing the gas clathrate; a reaction conduit which cools thus mixed and dissolved mixture therein while the mixture flows a reaction conduit; and a separator connected to the reaction conduit to separate the formed gas clathrate; the line mixer being positioned at the upstream side of the reaction conduit by at least one thereof; the line mixer being positioned in the reaction conduit by one or more thereof.

The line mixer is characterized in generating fine bubbles of the raw material gas.

A pressure regulating means for adjusting the line pressure is positioned at the downstream side of the line mixer.

A flow speed regulating means for adjusting the flow speed of fluid flowing through the line is positioned at the downstream side of the line mixer.

Hereinafter, there is described about gas hydrate that is one embodiment of the gas clathrate.

FIG. 18 shows outline of an embodiment of gas hydrate producing process according to Embodiment 5. The process uses natural gas as the raw material gas. The process uses natural gas as the raw material gas. The process for producing gas hydrate is outlined referring to FIG. 18.

Embodiment gives an improvement in the step (S3) that forms slurry gas hydrate from water and natural gas, whose improvement is to simplify the method and apparatus for efficiently producing hydrate. The detail of the improvement is described as follows.

FIG. 18 shows a process diagram of the Embodiment, giving main units. The units of Embodiment 5 are described referring to FIG. 18. The description given herein deals with gas clathrate, as an example, which is an object of Embodiment 5.

The gas clathrate producing apparatus of the Embodiment has a gas booster 1 that increases the pressure of raw material gas such as natural gas, raw material water pumps 3, 19 that feed the raw material water (“raw material water” referred in this specification is defined as either sole raw material water or the raw material water containing dissolved raw material gas), a first line mixer 5a that mixes the raw material water with the raw material gas to dissolve the raw material gas in the raw material water while the raw material water with the raw material flows in the first line mixer, a reaction conduit 7 that cools the mixture formed by the line mixer 5a to form the gas hydrate, a second line mixer 5b that is positioned in the reaction conduit 7 and that mixes the raw material water with the raw material gas to dissolve the raw material gas in the raw material water while the raw material water with the raw material gas flows in the second line mixer, a third line mixer 5c that is positioned in the reaction conduit 7 and at the downstream side of the second line mixer 5b and that mixes the raw material water with the raw material gas to dissolve the raw material gas in the raw material water while the raw material water with the raw material gas flow in the third line mixer, and a separator 9 that separates the gas clathrate formed in the reaction conduit 7, unreacted gas, and the raw material water from each other.

These units are connected to each other by pipes given in the drawing with solid lines with arrow marks. The pipelines, which feed the raw material gas to the respective line mixers 5a, 5b, 5c are equipped with the respective gas flow rate control valves 12a, 12b, 12c which adjust the gas flow rate.

The pipeline connecting the raw material pumps 3, 19 with the line mixer 5a is equipped with a flow speed control valve 14, which adjusts the flow speed of the raw material water.

The pipeline-feeding the raw material gas, after pressurized by the gas booster 1, to the separator 9 has a gas flow rate regulating valve 12d, which adjusts the feeding gas rate. The line recycling the surplus raw material gas in the separator 9 to the gas clathrate-forming line has a gas flow rate regulating valve 12e and a gas booster 2. According to the configuration, the gas flow rate control valves 12d, 12e are controlled, based on the signals transmitted from a pressure detector 10, which is mounted to the separator 9, and which detects the internal pressure of the separator 9, thus the internal pressure of the separator 9 is adjusted.

Further detailed description is given below for critical units in the line.

Each of the line mixers 5a, 5b, 5c in the Embodiment has a cylinder 11 of two-stage structure having a large diameter at the inlet and a small diameter at the exit, which is illustrated in FIG. 2. The cylinder 11 has blades 13 called the guide vanes in the large diameter section 11a, and has a plurality of impingers 15 in mushroom shape extending from the inner periphery of the cylinder toward the center thereof in the small diameter section 11b at the downstream side of the large diameter section 11a.

In such a type of line mixer 5, the raw material water that is fed by the raw material water pump 3 creates a swirling flow caused by the blades 13. The resulted strong centrifugal force pushes the raw material water outward, which raw material water then collides against the mushroom impingers 15 to receive stronger agitation. The raw material gas is caught by the vigorous agitation of the raw material water to form very fine bubbles, thus the raw material water and the raw material gas are mixed together. As a result, the contact area between the raw material gas and the raw material water increases in efficient dissolution of the raw material gas in the raw material water.

A curved pipe structures the reaction conduit 7. A chiller 17 cools the surface of the reaction conduit 7. The reaction conduit 7 makes it possible to cool from periphery efficiently. As a result, there is no need in cooling directly the gas and the raw material water by cooling coil or by other means, whose direct cooling is applied to the conventional method, thus the configuration of the facility becomes simple and compact.

The application of that type of reaction conduit 7 realizes to mix and dissolve the raw material gas and the raw material water in the line mixers 5a, 5b, 5c. This application makes it possible for the reaction conduit 7 to exclusively concentrate on cooling. That is, according to the conventional method described in the Patent document 1, the mixing, dissolving, and reaction cooling of the raw material gas and the raw material water are conducted in a tank-shape clathrate-forming vessel, so the mixing and dissolving action requires a determined volume of a space, which hinders the cooling solely from periphery of the reaction tank. On the contrary, according to the Embodiment, mixing and dissolving the raw material gas and the raw material water are separated from cooling the reaction, so the reaction step concentrate on cooling. As a result, the cooling by a simple configuration adapted to the process, described above.

The separator 9 separates gas hydrate, unreacted gas, and raw material water from each other. Applicable separator 9 includes decanter, cyclone, centrifugal separator, belt press, screw concentrator dehydrator, and rotary drier.

The process for producing gas clathrate using a facility of the Embodiment having configuration given above is described as follows.

The raw material gas, which increases the pressure to a predetermined level by the gas booster 1, is fed into the line mixer 5a via the gas flow rate control valve 12a. The raw material water, which increases the pressure to a predetermined level, is fed to the line mixer 5a via the flow speed control valve 14.

The raw material gas and the raw material water fed into the line mixer 5a are vigorously mixed together under the mechanism described before. The raw material gas becomes fine bubbles to enter the raw material water and to mix with the raw material water, thus enhancing the raw material gas to dissolve into the water.

The raw material water with the dissolved raw material gas, (containing undissolved fine bubbles), is fed to the reaction conduit 7, which is cooled by the chiller 17. Within the reaction conduit 7, the raw material gas is further mixed and dissolved into the raw material water by the line mixers 5b, 5c, then is fed to the separator 9.

At the beginning of the operation, the control valves 12d, 12e keep the internal pressure of the separator 9 at the condition of forming the hydrate, and the pressure in the reaction conduit connecting to the separator 9 is the equal or higher pressure. As a result, forming the gas clathrate begins in the reaction conduit.

The mechanism for forming clathrate in the reaction conduit 7 is described below.

The line mixer 5a mixes the raw material gas with the raw material water. The raw material gas becomes fine bubbles to dissolve in the raw material water, and the whole amount of the raw material water reaches an equilibrium concentration of the raw material gas.

When the raw material water reaches the equilibrium concentration, forming the gas clathrate begins, in case that the operating conditions are determined so as the pressure P in the reaction conduit 7 to become higher than the minimum pressure P₀ for forming clathrate, and so as the temperature T at each section in the reaction conduit 7 to become lower than the maximum temperature T₀ for forming clathrate. Forming the gas clathrate is accompanied with generating the corresponding reaction heat. Nevertheless, the temperature in the reaction conduit 7 is kept to be lower than the maximum temperature To for forming the clathrate by deriving the heat equivalent to the generated heat through the cooling by chiller 17. Excessive cooling results in solidifying the raw material water to hinder the smooth flow inside the reaction conduit 7. Accordingly, the cooling capacity of the chiller 17 is determined so as the raw material water to be the equal or higher than the solidification point of the raw material water.

Once the gas clathrate is formed, the dissolved gas concentration decreases, and then the raw material gas further dissolves into the raw material water until the equilibrium concentration is established again. When the concentration is the equal or higher than the equilibrium concentration, further quantity of gas clathrate is formed. In order to form a large amount of gas clathrate efficiently, it is required to increase the formed hydrate quantity within the period when the raw material water is flowing through the reaction conduit 7. In order to achieve such a procedure, it is required to make the actual quantity of the raw material gas, which dissolves into the raw material water, approach closely to the theoretical hydration number, to the utmost. In order to realize such a condition, it is required to establish an environmental condition that the raw material gas dissolves efficiently into the raw material water, when the raw material water becomes the equal or lower than the equilibrium concentration.

From this standing point of view, in this exemplary Embodiment, there are located the second and the third line mixers 5b, 5c on the way of the reaction conduit 7 to feed the raw material gas as the fine bubbles into the reaction conduit 7, resulting in dissolving the raw material gas into the water, actually. That is, even when the whole amount of the raw material gas, which becomes fine bubbles in the first line mixer 5a, is completely dissolved on the way of the reaction conduit 7, or even when the whole amount of the raw material gas, which is fine bubbles in the first line mixer 5a, is converted hydrate, or otherwise, even when the raw material gas, which becomes fine bubbles in the first line mixer 5a, exists as the bubbles as it is on the way of the reaction conduit 7, such respective bubbles join together and consolidate together to become other large-sized bubbles, while such bubbles flows through the reaction conduit 7. The large-sized bubbles end up in getting a smaller contact area with the raw material water. It makes it impossible to dissolve efficiently. In order to take the countermeasures against the problem, the raw material gas is fed again as fine bubbles on the way of the reaction conduit 7 for increase in the efficient dissolution of the raw material gas into the water.

The formed gas clathrate flows through the reaction conduit 7, and enters the separator 9 together with the raw material water and unreacted gas (unreacted gas does not exist when the total volume of the gas has converted to the clathrate).

Once the unreacted gas enters the separator 9, the internal pressure of the separator 9 increases. When the pressure detecting means 10 detects that the internal pressure of the separator 9 exceeded a predetermined level, the control means (not shown) controls the gas flow rate control valve 12e to force the surplus gas to return to the clathrate-forming line, thus adjusting the pressure in the separator 9 and the pressure in the reaction conduit 7.

In the separator 9, the gas clathrate, the unreacted gas, and the raw material water are separated from each other. The separated raw material water is recycled to the line mixer 5a via the pump 19.

On the other hand, the formed gas clathrate is taken out from the separator 9, and is fed to the succeeding process (S5 in FIG. 21 and the following processes).

In the separator 9, the water level is detected by a level gauge 21, and the water level in the separator 9 is controlled to be at the equal or higher than the predetermined level, in order to have an water-seal effect on the raw material water so as the gas not to enter the raw material water recycle line. The raw material water that is nor required for the water seal increases the pressure to a predetermined level by the raw material water pump 19, and is fed into the line mixer 5a.

As described above, in the exemplary Embodiment, there are applied a plurality of line mixers so that for the raw material gas to enhance dissolving into the raw material water. Consequently, such enhancement enables the clathrate to be formed efficiently.

According to the Embodiment, the reaction between the raw material gas and the raw material water is carried out while the reaction is forced to move through the conduit. So, the step of forming gas clathrate sends all the materials (formed gas clathrate, unreacted gas, and raw material water) to the separator 9. That is, there is no need in taking out only the formed gas clathrate, which is necessary for conventional method, tough. And the configuration of the facility becomes simple.

As described above, the line mixers continuously do dissolving the raw material gas in raw material water 5a, 5b, 5c in cylindrical shape, thus the dissolution is conducted in space-saving and efficient manner.

Furthermore, dissolving the raw material gas in the raw material water in the line mixers 5a, 5b, 5c, located separately from the clathrate-forming vessel, allows the process to use the pipe-shape reaction conduit 7, instead of the clathrate-forming vessel of large diameter. Consequently, Cooling means for cooling peripheral surface of the conduit become usable, as a simple and a compact one.

In addition, both dissolving the raw material gas in the line mixers 5a, 5b, 5c and gas clathrate in the reaction conduit 7 is done continuously so that the it makes it possible to produce gas clathrate efficiently and drastically.

The Embodiment shows one of the examples that two line mixers 5b, 5c are located at the downstream side of the line mixer 5a. The number of units of the line mixers positioned at the downstream side of the line mixer 5a, however, may be one, three or more. Alternatively, a plurality of line mixers may be located at the upstream side of the reaction conduit 7. The latter case is effective on the limitation, which is, the raw material gas has a limited amount against the raw material water, from the viewpoint of the mixing amount.

In the exemplary embodiment, pressure regulating means are not applied to the process between the line mixer 5a and the reaction conduit 7.

As shown in FIG. 19, however, a pressure regulating means 27 consisting of a pressure detector 23 and a regulating valve 25 may be located between the line mixer 5a and the pressure conduit 7.

The pressure regulating means 27 enables the pressure at the line mixer 5a side to be high, which further enhances the raw material water at the line mixer 5a to dissolve the raw material gas into the water. Furthermore, in order to enhance to dissolve the raw material gas into the raw material water, a holding section 29 as the flow speed regulating means may be located. The means are located at the downstream side of the line mixer 5a for decreasing the flow speed of fluid flowing through the line. It is shown in FIG. 20. Such holding section 29, enables the raw material gas, which became fine bubbles in the line mixer 5, to save time for dissolving into the raw material water. This enhances to dissolve the raw material gas.

As one example of the holding section 29, a tank is followed up, which has a determined volume.

The above-given description on the Embodiment does not express the details of the temperature and the pressure in the individual steps. In the exemplary example, one may be a case, in FIG. 21. The temperature and the pressure at the_respective steps are selected to be the respective optimum values.

Further examples of line mixer include that such as Venturi type, which has a thinning portion in the middle of the cylinder to generate negative pressure for sucking and mixing the raw material gas, or that in cone or truncated cone shape to create swirling flow to conduct mixing of gas and liquid. An example of the latter type mixer is a swirling type fine bubble generator disclosed in Japanese Patent Laid-Open No. 2000-447. In short, the line mixer according to the present invention includes broadly the unit which is located in the line and which is capable of mixing gas with the liquid continuously.

According to the above-given Embodiment, the reaction conduit 7 is single. The reaction conduit 7 may be, however, formed by a plurality, each having the same number of line mixers. Alternatively, it may be a case, the number of the units of the line mixers located in the individual reaction conduits different from each other. Furthermore, the reaction conduit may be branched. And a plurality of the units of the line mixers may be positioned in the reaction conduit before branching, without locating the line mixer in each branched conduit after branching. Otherwise, the same number of the line mixer may be located or the different number of the line mixers may be located at each branched conduit.

In the above-mentioned exemplary embodiment, there has been described the natural gas consisting mainly of methane gas as the raw material gas. As other applicable examples of the raw material gas, there are followed up ethane, propane, butane, krypton,.xenon, and carbon dioxide.

The above-given Embodiment does not specify the kind of raw material water. Examples of the raw material water include plain water, seawater, and antifreeze liquid. Instead of the raw material water, liquid host material or host material solution may be applied to the process.

### Embodiment 6

The method for transporting gas according to the Embodiment 6 has the steps of: mixing a raw material water with a raw material gas and dissolving the raw material gas in the raw material water in the route for producing the gas hydrate; cooling the mixed and dissolved mixture while the mixture flows through a reaction conduit to form the gas hydrate; successively storing the formed gas hydrate in a transportation tank connected to the reaction conduit; and disconnecting the transportation tank from the reaction conduit to transport to a destination.

The method for transporting gas according to the Embodiment further has the step of concentrating the formed gas hydrate or the step of separating the formed gas hydrate from the raw material water.

The apparatus for transporting gas through the reaction between a raw material water and a raw material gas to form a gas hydrate, according to the Embodiment 6, has: a line mixer which mixes the raw material water with the raw material gas and dissolves the raw material gas in the raw material water in the route for producing the gas hydrate; a reaction conduit which cools the mixed and dissolved mixture; and a transportation tank that is detachably connected with the reaction conduit and that stores the gas hydrate formed in the reaction conduit, and which is disconnected from the reaction conduit after filled with the gas hydrate for transporting to a destination.

The apparatus for transporting gas according to the Embodiment further has a concentration unit, which concentrates the formed gas hydrate, or a separator, which separates the formed gas hydrate from the raw material water.

FIG. 22 shows a process flow diagram for realizing the method, showing main units. The units according to the Embodiment are described below referring to FIG. 22.

The apparatus of the Embodiment has a gas booster 1 that increases the pressure of raw material gas such as natural gas, a raw material water pump 3 that feeds the raw material water stored in a raw material tank 2 to a line mixer 5 (described later), the line mixer 5 that mixes the raw material water with the raw material gas to dissolve the raw material gas in the raw material water, a reaction conduit 7 that cools the mixture formed in the line mixer 5 while the mixture flows to form the gas hydrate, a chiller 17 that cools the reaction conduit 7, and a transportation tank 9 that is detachably connected to the reaction conduit 7 and that stores the gas hydrate formed in the reaction conduit 7.

Such units are connected to each other by pipes given in the drawing with solid lines with arrow marks. The pipeline that feeds the raw material gas to the line mixer 5 is equipped with a pressure detector 6 and a valve 4 that is actuated based on the detected value on the pressure detector 6.

There is a pipeline between the transportation tank 9 and the upstream side of the line mixer 5 to recycle the gas in the transportation tank 9 to the line mixer 5. On the pipeline, a valve 10 is mounted, which valve 10 is controlled by the signals transmitted from a pressure detector 8 attached to the transportation tank 9. Furthermore, the pipeline has a gas booster 12.

There is a pipeline between the transportation tank 9 and the raw material water tank 2 to recycle the raw material water to the raw material tank 2, and the pipeline is further provided with a raw material water pump 19.

Further detailed description is given below for critical units in the line.

The line mixer 5 in the Embodiment 6 has a cylinder 11 of two-stage structure giving a large diameter at inlet and a small diameter at exit, which is illustrated in FIG. 2. The cylinder 11 has blades 13 called the guide vanes in the large diameter section 11a, and has a plurality of impingers 15 in mushroom shape extending from the inner periphery of the cylinder toward the center in the small diameter section 11b at downstream side of the large diameter section 11a.

In such a type of line mixer 5, the raw material water, which is fed by the raw material water pump 3, creates a swirling flow caused by the blades 13. The resulted strong centrifugal force pushes the raw material water outward, which raw material water then collides against the mushroom impingers 15 to receive stronger agitation. The raw material gas is caught by the vigorous agitation of the raw material water to form very fine bubbles, thus the raw material water and the raw material gas are mixed together. As a result, the contact area between the raw material gas and the raw material water increases in dissolving efficiently the raw material gas in the raw material water.

A curved pipe structures the reaction conduit 7. The chiller 17 cools the surface of the reaction conduit 7. The reaction conduit 7 makes it possible to cool from periphery efficiently. As a result, there is no need in cooling directly the gas and the raw material water by cooling coil or by other means, whose direct cooling is applied to the conventional method, thus the configuration of the facility becomes simple and compact.

The application of type of reaction conduit 7 realizes by mixing and dissolving the raw material gas and the raw material water in the line mixer 5, in advance. And the reaction conduit 7 enables the process to exclusively concentrate on cooling. That is, according to the conventional method described in the Patent document 1, the mixing, dissolving, and reaction cooling of the raw material gas and the raw material water are conducted in a tank-shape pressure vessel, so the mixing and dissolving action requires a determined volume of a space, which hinders the cooling solely from periphery of the reaction tank. On the contrary, according to the Embodiment, the mixing and dissolving of the raw material gas and the raw material water are separated from the reaction cooling, so the reaction step concentrate on cooling. As a result, the cooling with simple configuration described above is applied to the process.

The transportation tank 9 is detachably connected with the reaction conduit 7. When a predetermined amount of hydrate is fed into the transportation tank 9, the tank 9 is disconnected from the reaction conduit 7, and is transported by a transporting means such as truck 20 (refer to FIG. 1). At the inlet of the transportation tank 9, a concentrator utilizing the difference in fluid density may be attached. The concentrator enables the gas hydrate to enrich by passing therethrough, thus feeding the concentrated gas hydrate into the transportation tank 9. Furthermore, a unit such as a decanter, a cyclone, a centrifugal separator, a belt press, a screw concentrator and a dehydrator, and a rotary drier may be applied to separate the gas hydrate from the raw material water. Through the unit, the gas hydrate separated from the raw material water may be fed into the transportation tank 9.

The transportation tank 9 transports the gas hydrate under the condition of the equal or lower temperature of the equilibrium determined by the pressure. For example, the equilibrium temperature for the case of methane hydrate is: −80° C. or lower at atmospheric pressure, 0° C. or lower at 25 atm, and 10° C. or lower at 80 atm.

Consequently, the transportation tank 9 is required to have a pressure-resistant and adiabatic structure to endure the above-given pressures and to assure the above-determined equilibrium temperatures. Alternatively, a refrigerator may be mounted to the transportation tank for long distance transportation.

Although the equilibrium temperature of methane hydrate is −80° C. at atmospheric pressure, it is known that methane hydrate is stored at higher temperatures range from −20° C. to −10° C. The reason is that gas is released from the surface of methane hydrate by dissociation to form ice shell on the surface thereof, whose ice shell acts as a protective casing to prevent internal hydrate from dissociating (the phenomenon is named for the “self-sustainability”). Therefore, it maybe a case, the transportation under the condition of such an equilibrium temperature or higher, the transportation is available.

The method for transporting gas using the apparatus according to the Embodiment having the configuration described above is written up as follows.

The pressure of the raw material gas increases to be a determined level by the gas booster 1. The pressure of the raw material water increases to a determined level by the raw material water pump 3. Both the pressurized raw material gas and the pressurized raw material water are fed into the line mixer 5.

The gas flow rate control valve 4 controls the pressurized raw material gas. So, the raw material gas enters the line mixer 5 at a determined flow rate. The raw material gas and the raw material water fed into the line mixer 5 are vigorously mixed together under the control of the mechanism described before. The raw material gas becomes fine bubbles to enter the raw material water and to mix with the raw material water, thus enhancing the raw material gas to dissolve into the raw material water. The raw material water having the dissolved raw material gas, (which contains the undissolved fine bubbles), is fed into the reaction conduit 7, where the mixture is cooled by the chiller 17 to form the gas hydrate.

The mechanism for forming the hydrate in the reaction conduit 7 is described below. That is, the line mixer 5 mixes the raw material gas with the raw material water. The raw material gas becomes fine bubbles to dissolve in the raw material water. And, the whole amount of the raw material water reaches an equilibrium concentration of the raw material gas.

When the raw material water reaches the equilibrium concentration, gas hydrate begins to be formed, because the operational conditions are determined so as the pressure P in the reaction conduit 7 to become higher than the minimum pressure P₀ for forming hydrate, and so as the temperature T at each section in the reaction conduit 7 to become lower than the maximum temperature T₀ for forming hydrate. Forming the gas hydrate is accompanied with generation of reaction heat. Nevertheless, the temperature in the reaction conduit 7 is kept to be lower level than the maximum temperature T₀ for forming hydrate by deriving the heat, which is equivalent to the generated heat during cooling by chiller 17. Additionally, excessive cooling causes the raw material water to solidify the resulting in hindering the smooth flow inside the reaction conduit 7. Accordingly, the cooling capacity of the chiller 17 is determined so as the raw material water not to become the equal or lower than the solidification point of the water.

Once the gas hydrate is formed, the concentration of the dissolved gas decreases, and then the raw material gas further dissolves into the raw material water until the equilibrium concentration is established again. When the concentration becomes the equal or higher than the equilibrium concentration, further quantity of gas hydrate is formed.

Thus formed gas hydrate flows through the conduit, together with the unreacted gas and the raw material water, to enter the transportation tank 9 in a slurry state. The unreacted water is discharged from the bottom of the tank by the raw material water pump 19. Instead of using the raw material water pump 19, it may be a case, the unreacted water is taken out by natural gravity from the bottom part of the tank.

As mentioned above, the transportation tank filled with the gas hydrate and the unreacted water is transported to the destination, using a trailer or the like. After the transportation tank arrives at the destination, the tank is depressurized to drop to atmospheric pressure, resulting in releasing the raw material gas contained in the gas hydrate. At the same time, it may be a case, a heater is located in the transportation tank 9 to heat the hydrate.

On the way of the gas emission pipe, it may be a case, a dehumidifier is located, if necessary, to remove the water content from the raw material gas.

According to the Embodiment, as described above, the raw material gas and the raw material water are reacted mutually, while the raw material gas and the raw material water move through the conduit. Therefore, the step of forming gas hydrate sends all of the materials (the formed gas hydrate, the unreacted gas, and the raw material water) to the transportation tank 9. That is, there is no need in taking out only the formed gas hydrate, and such a fact enables the configuration of the facility to be simplified. As described above, the line mixer 5 in a cylindrical shape makes it possible to dissolve continuously the raw material gas into the raw material water continuously, and the reaction conduit 7 makes it possible to form continuously the gas hydrate. Consequently, the production efficiency of the gas hydrate remarkably improves, and the efficient transportation of the gas hydrate realizes.

And, in the exemplary embodiment mentioned above, there has no explicit description about the respective temperature and the respective pressure. But, the optimal values of the respective temperature and the respective pressure are selected, taking into consideration the temperature and the pressure in the respective process.

In the exemplary embodiment, as mentioned above, the description is written, based on natural gas, which has methane as a main ingredient in the raw material gas. However, as other examples of the raw material gas, there are usable ones, these are, ethane, propane, butane, krypton, xenon, and carbon dioxide and the like.

Furthermore, as other examples of the line mixers, it may be a case, a line mixer such as Venturi type, which has a thinning portion in the middle of the cylinder to generate the negative pressure for sucking and mixing the raw material gas, or a line mixer such as that in a cone shape or that in a truncated cone shape, in order to create swirling the flow stream, which enables the gas to mix with the liquid. An example of the latter type mixer is a swirling type fine bubble generator, which is disclosed, in Japanese Patent Laid-Open No. 2000-447. In short, the line mixer according to the Embodiment is broadly defined as the unit that is located in the line and that mixes gas with liquid continuously.

According to the above-given Embodiment, it may be a case, the reaction conduit 7 is a single curbed pipe, as one of the examples. And, it may be a case, the reaction conduit 7, is a plurality of curbed pipes, or is a straight pipe instead of curved pipe.

The above-given Embodiment does not express explicitly the kinds of the raw material water. As examples of the raw materials, there are followed up water include plain water, seawater, and antifreeze liquid.

1. A method for producing gas clathrate, comprising the steps of;
mixing and dissolving step of mixing a raw material liquid with a raw material gas in a production- line of gas clathlate and dissolving the raw material gas into the raw material liquid;
forming step of cooling the gas-dissolved raw material liquid while forcing the gas-dissolved raw material liquid to flow through a reaction conduit to form the gas clathlate.

2. The method of claim 1, wherein the mixing and dissolving step comprises coninuously dissolving the raw material gas in the form of fine bubbles into the raw material liquid.

3. The method of claim 1, wherein
the mixing and dissolving step comprises without using a reaction vessel, mixing a raw material liquid with a raw material gas in a production line of gas clathlate and dissolving the raw material gas into the raw material liquid;
the forming step comprises without using a reaction vessel, cooling the gas-dissolved raw material liquid while forcing the gas-dissolved raw material liquid to flow through the reaction conduit to form the gas clathrate.

4. The method of claim 3, wherein the mixing and dissolving step comprises continuously dissolving the raw material gas in the form of fine bubbles into the raw material liquid.

5. The method of claim 1, further comprising the steps of sending the formed gas clathrate together with the unreacted raw material gas and the raw material liquid through the reaction conduit to a separator.

6. The method of claim 1, further comprising the steps of:
sending the formed gas clathrate together with the unreacted raw material gas and the raw material liquid through the reaction conduit to a separator; and
subjecting a slurry including the gas clathrate, the unreacted raw material gas and the raw material liquid to a separation and dewatering treatment by the separator to form a high concentration slurry or a solid clathrate.

7. The method of claim 5, wherein the mixing of the raw material liquid and the raw material gas is continuously performed by using a line mixer.

8. The method of claim 6, wherein the mixing of the raw material liquid and the raw material gas is continuously performed by using a line mixer.

9. The method of claim 1, wherein the mixing and dissolving step comprises mixing a raw material liquid with a raw material gas by using a line mixer and dissolving the raw material gas into the raw material liquid.

10. A method for producing gas clathrate, comprising the steps of:
mixing and dissolving step of mixing a raw material liquid with a raw material gas in a production line of gas clathrate and dissolving the raw material gas into the raw material liquid;
forming step of cooling the gas-dissolved raw material liquid while forcing the gas-dissolved raw material liquid to flow through a-reaction conduit to form the gas clathrate; wherein,
the mixing and dissolving step comprises mixing a raw material liquid with a raw material gas by using a line mixer which is different form a reaction tank and dissolving the raw material gas into the raw material liquid; and wherein,
the forming step comprising forcing the gas-dissolved raw material liquid to flow through a pipe-shaped reaction conduit which is different from a reaction tank and cooling the surface of the reaction conduit to form the gas clathrate.

11. A method for producing gas clathrate, comprising the steps of:
mixing and dissolving step of mixing a raw material liquid with a raw material gas in a production line of gas clathrate and dissolving the raw material gas into the raw material liquid;
forming steps of cooling the gas-dissolved raw material liquid while forcing the gas-dissolved raw material liquid to flow through a reaction conduit to form the gas clathrate;
sending the formed gas clathrate together with the unreacted raw material gas and the raw material liquid through the reaction conduit to a separator; and
separating the mixture into the gas clathrate, the unreacted raw material gas and the raw material liquid; wherein,
the mixing and dissolving step comprising mixing a raw material liquid with a raw material gas by using a line mixer which is different from a reaction tank and dissolving the raw material gas into the raw material liquid; and wherein,
the forming step comprising forcing the gas-dissolve raw material liquid to flow through a pipe-shaped reaction conduit which is different from a reaction tank and cooling the surface of the reaction conduit to form the gas clathrate.

12. The method of claim 9, further comprising the step of providing a pressure regulating means between the line mixer and the reaction conduit and adjusting a fluid pressure such that the pressure of the line mixer side becomes higher than the pressure of the reaction conduit side.

13. The method of claim 10, further comprising the step of providing a pressure regulating means between the line mixer and the reaction conduit and adjusting a fluid pressure such that the pressure of the line mixer side becomes higher than the pressure of the reaction conduit side.

14. The method of claim 11, further comprising the step of providing a pressure regulating means between the line mixer and the reaction conduit and adjusting a fluid pressure such that the pressure of the line mixer side becomes higher than the pressure of the reaction conduit side.

15. The method of claim 9, further comprising the step of providing a flow speed regulating means at downstream side of the line mixer and adjusting a flow speed of fluid to reduce the flow speed flowing through the line.

16. The method of claim 10, further comprising the step of providing a flow speed regulating means at downstream side of the line mixer and adjusting a flow speed of fluid to reduce the flow speed flowing through the line.

17. The method of claim 11, further comprising the step of providing a flow speed regulating means at downstream side of the line mixer and adjusting a flow speed of fluid to reduce the flow speed flowing through the line.

18. The method of claim 9, further comprising the step of recycling the unreacted raw material gas and the raw material liquid, which were separated in the separating step.

19. The method of claim 10, further comprising the step of recycling the unreacted raw material gas and the raw material liquid, which were separated in the separating step.

20. The method of claim 11, further comprising the step of recycling the unreacted raw material gas and the raw material liquid, which were separated in the separating step.

21. The method of claim 19, further comprising the step of controlling a liquid level in the separator to give the raw material liquid seal effect so as the gas not to enter the raw material liquid recycle line.

22. The method of claim 20, further comprising the step of controlling a liquid level in the separator to give the raw material liquid seal effect so as the gas not to enter the raw material liquid recycle line.

23. The method of claim 19, further comprising the step of supplying a raw material gas, whose pressure increases by a gas booster, to the separator.

24. The method of claim 20, further comprising the step of supplying a raw material gas, whose pressure increases by a gas booster, to the separator.

25. A method for producing gas clathrate, comprising the steps of:
mixing and dissolving step of mixing a raw material liquid with a raw material gas in a production line of gas clathrate and dissolving the raw material gas into the raw material liquid;
forming step of cooling the gas-dissolved raw material liquid while forcing the gas-dissolved raw material liquid to flow through a reaction conduit to form the gas clathrate; wherein,
the mixing and dissolving step and the forming step is separately performed.

26. The method of claim 25, wherein
the mixing and dissolving step comprises mixing a raw material liquid with a raw material gas by using a line mixer in a production line of gas clathrate and dissolving the raw material gas into the raw material liquid; and
the forming step comprises forcing the gas-dissolved raw material liquid to flow through a pipe-shaped reaction conduit and cooling the surface of the reaction conduit to form the gas clathrate.

27. The method of claim 9, wherein the mixing and dissolving step comprises mixing the raw material liquid and the raw material gas by using the line mixer to convert the raw material gas to fine bubbles and dissolving the fine bubbles into the raw material liquid.

28. The method of claim 10, wherein the mixing and dissolving step comprises mixing the raw material liquid and the raw material gas by using the line mixer to convert the raw material gas to fine bubbles and dissolving the fine bubbles into the raw material liquid.

29. The method of claim 11, wherein the mixing and dissolving step comprises mixing the raw material liquid and the raw material gas by using the line mixer to convert the raw material gas to fine bubbles and dissolving the fine bubbles into the raw material liquid.

30. The method of claim 9, wherein the mixing and dissolving step comprises agitating the raw material liquid, involving the raw material gas in the agitated raw material liquid to convert the raw material gas into fine bubbles, and dissolving the fine bubbles into the raw material liquid.

31. The method of claim 10, wherein the mixing and dissolving step comprises agitating the raw material liquid, involving the raw material gas in the agitated raw material liquid to convert the raw material gas into fine bubbles, and dissolving the fine bubbles into the raw material liquid.

32. The method of claim 11, wherein the mixing and dissolving step comprises agitating the raw material liquid, involving the raw material gas in the agitated raw material liquid to convert the raw material gas into fine bubbles, and dissolving the fine bubbles into the raw material liquid.

33. The method of claim 1, wherein the forming step comprises converting the whole amount of the material gas dissolved in the mixing and dissolving step into the gas clathrate.

34. The method of claim 1, wherein, in the forming step, flow rate of the raw material liquid, pressure of the raw material liquid, flow rate of the raw material gas, pressure of the raw material gas, cooling capacity, reaction conduit length and reaction conduit diameter are determined to be fallen within a range so that the pressure P at exit of the reaction conduit becomes higher than the minimum pressure P₀ for forming the gas clathrate, so that the temperature T in the reaction conduit becomes lower than the maximum temperature T₀ for forming the gas clathrate, and so that the raw material gas dissolved in the mixing and dissolving step derives all of formation heat that is accompanied by converting the whole amount of the dissolved raw material gas into the gas clathrate.

35. The method of claim 1, further comprising the step of changing a particle size of the gas clathrate by changing at least one selected from the group consisting of flow speed of the raw material liquid that flows through the reaction conduit, and supply amount of the raw material gas.

36. The method of claim 1, wherein the reaction conduit comprises a plurality of conduits, and wherein
the method for producing the gas clathrate further comprises a step of changing a particle size of the gas clathrate formed in the respective reaction conduits in the forming step by changing at least one selected from the group consisting of flow speeds of the raw material liquid that flows through each of a plurality of the reaction conduits and flow rate of the raw material gas being fed to the respective reaction conduits.

37. The method of claim 1, further comprising the steps of:
separating the formed gas clathrate by a separator connected with the reaction conduit;
detecting pressure in the separator; and
controlling the pressure in the separator by adjusting at least one selected from the group consisting of flow rate of the raw material gas being fed to the mixing and dissolving step and flow speed of the raw material liquid in the forming step, based on the pressure detected in the pressure detecting step.

38. The method of claim 1, further comprising the steps of:
separating the formed gas clathrate by a separator connected with the reaction conduit;
further mixing and dissolving step of dissolving the raw material gas into the raw material liquid after the mixing and dissolving step, and before the forming step of the gas clathrate or on the way of the forming step of the gas clathrate.

39. The method of claim 1, further comprising the steps of:
successively storing the formed gas clathrate in a transportation tank connected to the reaction conduit; and
dismounting the transportation tank from the reaction conduit to transport to a destination.

40. The method of claim 39, further comprising the step of concentrating the formed gas clathrate or the step of separating the formed gas clathrate from the raw material liquid.

41. An apparatus for producing gas clathrate, comprising:
a line mixer for mixing a raw material liquid with raw material gas and dissolving the raw material gas in the raw material liquid in a production line of the gas clathrate; and
a reaction conduit, where forcing the gas-dissolved raw material liquid to flow, for forming the gas clathrate by cooling the gas-dissolved raw material liquid.

42. The apparatus of claim 41, the line mixer further comprising means for generating fine bubbles of the raw material gas.

43. The apparatus of claim 41, further comprising pressure regulating means at downstream side of the line mixer for adjusting line pressure.

44. The apparatus of claim 42, further comprising pressure regulating means at downstream side of the line mixer for adjusting line pressure.

45. The apparatus of claim 41, further comprising flow speed regulating means for adjusting flow speed of the fluid that flows through the line.

46. The apparatus of claim 42, further comprising flow speed regulating means for adjusting flow speed of the fluid that flows through the line.

47. The apparatus of claim 43, further comprising flow speed regulating means for adjusting flow speed of the fluid that flows through the line.

48. The apparatus of claim 41, further comprising the apparatus other than a tank-shape pressure vessel for mixing a raw material liquid with a raw material gas and dissolving the raw material gas in the raw material liquid and for cooling the gas-dissolved raw material liquid.

49. The apparatus of claim 48, wherein the line mixer further comprising means for generating fine bubbles of the raw material gas.

50. The apparatus of claim 48, further comprising a pressure regulating means at downstream side of the line mixer to adjust line pressure.

51. The apparatus of claim 49, further comprising a pressure regulating means at downstream side of the line mixer to adjust line pressure.

52. The apparatus of claim 48, further comprising flow speed regulating means to adjust flow speed of fluid flowing through the line.

53. The apparatus of claim 49, further comprising flow speed regulating means to adjust flow speed of fluid flowing through the line.

54. The apparatus of claim 41, further comprising a separator for separating the gas clathrate, the unreacted raw material gas and the raw material liquid, which are formed in the reaction conduit.

55. The apparatus of claim 54, wherein the line mixer comprises means for generating fine bubbles of the raw material gas.

56. The apparatus of claim 54, wherein the separator comprises at least one selected from the group consisting of a decanter, cyclone, a centrifugal separator, a belt press, a screw concentrator dehydrator, and a rotary drier.

57. The apparatus of claim 41, further comprising:
gas flow rate regulating means for adjusting flow rate of the feeding raw material gas;
gas pressure regulating means for adjusting the raw material gas pressure;
raw material liquid flow rate regulating means for adjusting flow rate of the feeding raw material water;
raw material liquid pressure regulating means to adjust pressure of the raw material liquid;
cooling unit for cooling the reaction conduit; and
pressure regulating means for adjusting the pressure in the reaction conduit; wherein
the gas flow rate regulating means, the gas pressure regulating means, the raw material liquid flow rate regulating means, the raw material pressure regulating means, cooling capacity of the cooling unit, length of the reaction conduit, and diameter of the reaction conduit are determined to be controlled so that the whole amount of the raw material gas supplied into the line mixer are converted into the gas clathrate.

58. The apparatus of claim 41, further comprising:
gas flow rate regulating means for adjusting flow rate of the feeding raw material gas;
gas pressure regulating means for adjusting the raw material gas pressure;
raw material liquid flow rate regulating means for adjusting flow rate of the feeding raw material liquid;
raw material liquid pressure regulating means for adjusting pressure of the raw material liquid;
a cooling unit for cooling the reaction conduit; and
pressure regulating means for adjusting the pressure in the reaction conduit; wherein
the gas flow rate regulating means, the gas pressure regulating means, the raw material liquid flow rate regulating means, the raw material liquid pressure regulating means, cooling capacity of the cooling unit, length of the reaction conduit, and diameter of the reaction conduit are determined to be controlled so that the pressure P at exit of the reaction conduit becomes higher than the minimum pressure P₀ for forming the gas clathrate, the temperature T in the reaction conduit becomes lower than the maximum temperature T₀ for forming the gas hydrate, and the raw material gas supplied into the line mixer derives the whole amount of the formation heat, which is accompanied by converting all of the dissolved raw material into the gas clathrate.

59. The apparatus of claim 57, further comprising:
a pressure detector for detecting pressure at exit of the reaction conduit, to adjust at least one selected from the group consisting of the gas flow rate regulating means and the raw material liquid flow rate regulating means, when pressure value detected by the pressure detecting means exceeds a predetermined level.

60. The apparatus of claim 58, further comprising:
a pressure detector for detecting the pressure at exit of the reaction conduit to adjust at least one selected from the group consisting of the gas flow rate regulating means and the raw material liquid flow rate regulating means, when pressure value detected by the pressure detecting means exceeds a predetermined level.

61. The apparatus of claim 57, wherein the line mixer comprises means for generating fine bubbles of the raw material gas.

62. The apparatus of claim 58, wherein the line mixer comprises means for generating fine bubbles of the raw material gas.

63. The apparatus of claim 41, further comprising a flow speed controlling means for changing the flow speed of the raw material liquid that flows through the reaction conduit.

64. The apparatus of claim 41 having a plurality of conduits, further comprising:
flow speed controlling means for changing the flow speed of the raw material water that flows through the plurality of reaction conduits, wherein the flow speed controlling means controls so as the respective flow speed of the raw material liquid flowing through the individual reaction conduit to be different from each other.

65. The apparatus of claims 41, further comprising gas flow rate regulating means for adjusting flow rate of the raw material gas that is fed into the line mixer.

66. The apparatus of claim 41, wherein
the apparatus has a plurality of the line mixers and a plurality of the conduits;
the plurality of the line mixers have raw materials gas flow rate regulating means respectively, wherein the raw material gas flow rate regulating means control the flow rate of the raw material gas so that the flow rate of the raw material gas that flows through the individual reaction conduit is different from each other.

67. The apparatus of claim 54, further comprising:
gas flow rate regulating means for adjusting material gas flow rate to be supplied;
pressure detecting means for detecting pressure in the separator;
control means for adjusting at least one selected from the group consisting of the gas flow rate of the flow rate regulating means and the flow speed of the raw material liquid flow speed regulating means, based on the pressure value detected by the pressure detecting means.

68. The apparatus of claim 67, wherein the line mixer further comprises means for generating fine bubbles of the raw material gas.

69. The apparatus of claim 54, wherein at least one piece of the line mixer is located on upstream side of the reaction conduit, simultaneously with locating a single of the line mixer or a plural of the line mixer on the way of the reaction conduit.

70. The apparatus of claim 69, the line mixer further comprising means for generating fine bubbles of the raw material gas.

71. The apparatus of claim 69, further comprising a pressure regulating means at downstream side of the line mixer to adjust line pressure.

72. The apparatus of claim 70, further comprising a flow speed regulating means for adjusting flow speed of the fluid that flows through the line.

73. The apparatus of claim 69, further comprising flow speed regulating means for adjusting flow speed of the fluid that flows through the line.

74. The apparatus of claim 70, further comprising flow speed regulating means for adjusting flow speed of the fluid that flows through the line.

75. The apparatus of claim 41, further comprising:
a transportation tank, which is detachably connected with the reaction conduit, which stores the gas clathrate formed in the reaction conduit, and which is dismounted from the reaction conduit after the transportation tank is filled with the gas clathrate for transporting the gas clathrate to destination.

76. The apparatus of claim 75, further comprising at least one selected from the group consisting of a concentration unit for concentrating the produced gas hydrate and a separator for separating the produced gas hydrate from the raw material liquid.
